(19) United States
(12) Patent Application Publication
(10) Pub. No.: US 2013/0131073 A1
(43) Pub. Date: May 23, 2013

(54) FUSED-IMIDAZOYL COMPOUNDS USEFUL AS ANTIMICROBIAL AGENTS

(71) Applicants: Gay Lynn Miesel, New York, NY (US); Srikanth Venkatraman, Edison, NJ (US)

(72) Inventors: Gay Lynn Miesel, New York, NY (US); Srikanth Venkatraman, Edison, NJ (US)

(21) Appl. No.: 13/813,485

(22) PCT Filed: Jul. 28, 2011
(86) PCT No.: PCT/US11/45646

§ 371 Date: Jan. 31, 2013

Related U.S. Application Data
(60) Provisional application No. 61/370,169, filed on Aug. 3, 2010.

(51) Int. Cl. A61K 31/4985

(52) U.S. Cl. CPC A61K 31/4985
USPC 514/249

(57) ABSTRACT

Disclosed are methods of treating microbial infections, comprising administering a compound of Formula (I) Formula I wherein R¹, R², R³ and R⁴ are defined herein.

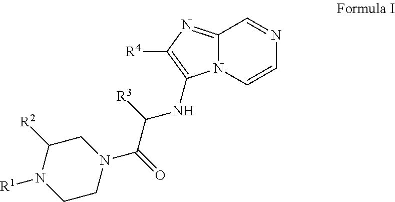

### FIELD OF THE INVENTION

The present invention relates to methods of using fused-imidazoyl compounds useful as antimicrobial agents.

### BACKGROUND OF THE INVENTION

Resistance to antibiotics is a growing medical concern as infections caused by resistant organisms are difficult to treat. Resistance is particularly problematic among bacterial pathogens such as Enterococcus faecium, Staphylococcus aureus, Mycobacterium tuberculosis, Pseudomonas aeruginosa, Klebsiella pneumoniae, and Acinetobacter baumannii where resistance to multiple antibiotics is often observed. Consequently, there is a need to develop new antibiotics to treat infections caused by drug resistant microbes.

### SUMMARY OF THE INVENTION

The present invention provides methods of treating microbial infections, comprising administering to a patient in need of such treatment a compound of Formula I:

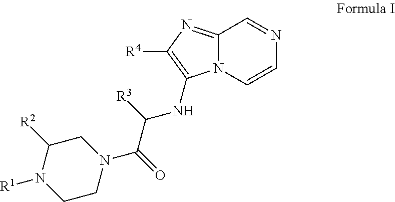

or a pharmaceutically acceptable salt, solvate, hydrate, ester, prodrug, or stereoisomer thereof, wherein:

R¹ is methyl,

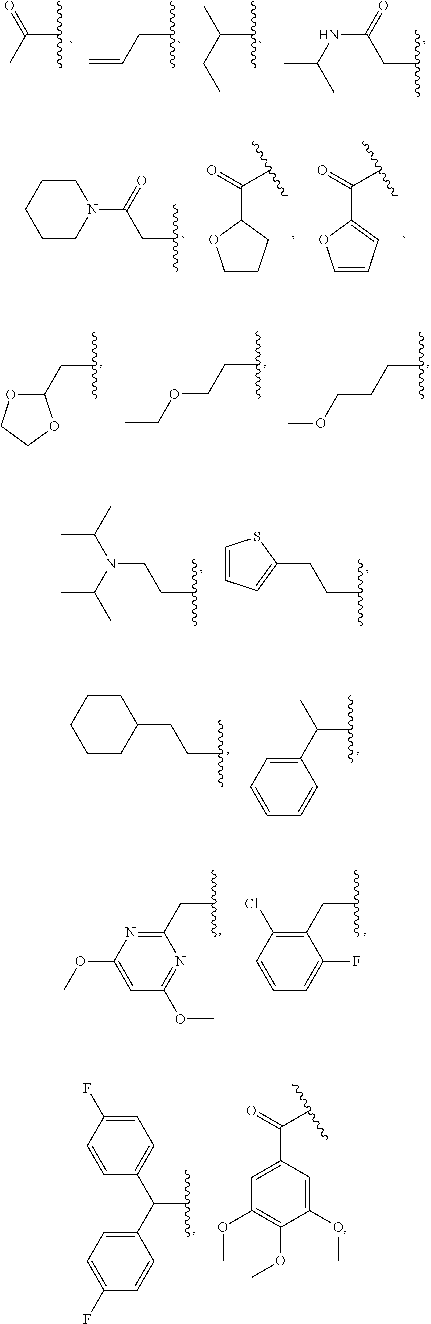

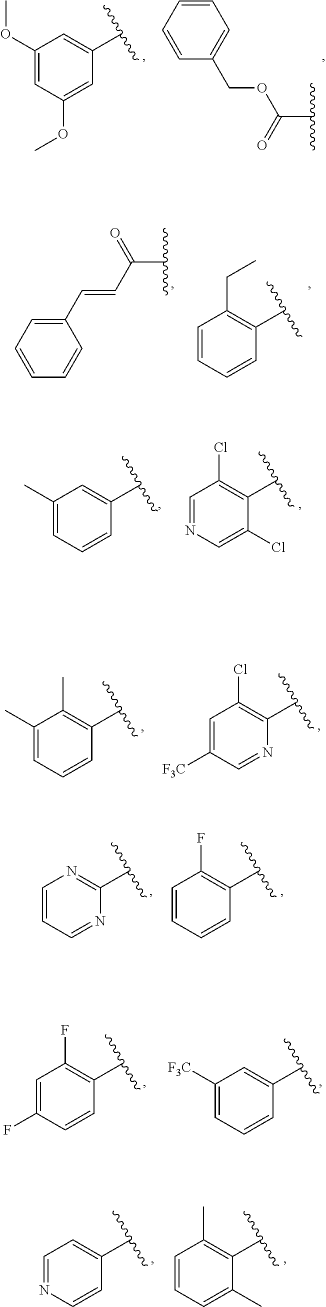

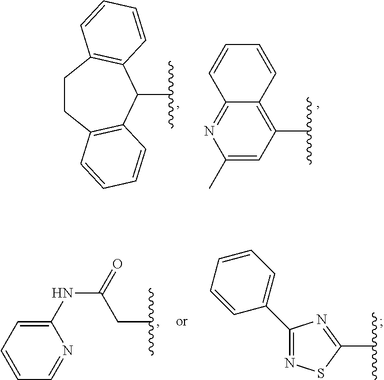

R² is hydrogen or methyl;

R³ is hydrogen or methyl; and

R⁴ is

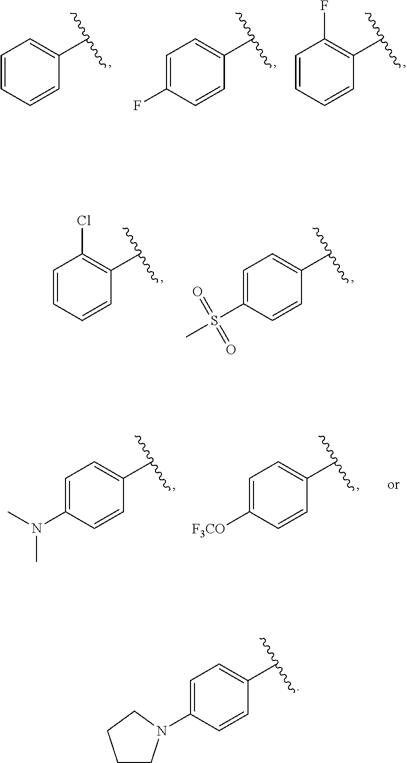

### DETAILED DESCRIPTION

In one embodiment of the present invention, R² is hydrogen.

In one embodiment of the present invention, R² is methyl. In one embodiment of the present invention, R³ is hydrogen. In a class of this embodiment, R² is hydrogen. In another class of this embodiment, R² is methyl.

In one embodiment, R³ is methyl. In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

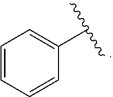 [SMILES: *c1ccccc1]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

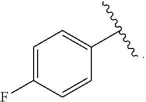 [SMILES: *c1ccc(F)cc1]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

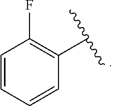 [SMILES: *c1ccccc1F]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

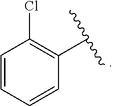 [SMILES: *c1ccccc1Cl]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

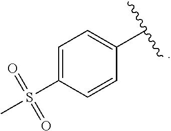 [SMILES: *c1ccc(S(C)(=O)=O)cc1]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

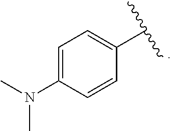 [SMILES: *c1ccc(N(C)C)cc1]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, R⁴ is

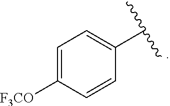 [SMILES: *c1ccc(OC(F)(F)F)cc1]

In a class of this embodiment, R² is hydrogen. In a subclass of this class, R³ is hydrogen. In another subclass of this class, R³ is methyl. In a subclass of this class, R³ is hydrogen. In another subclass of this class, R³ is methyl.

In another class of this embodiment, R² is methyl.

In one embodiment of the present invention, R⁴ is

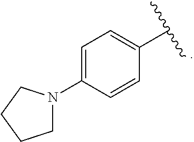 [SMILES: *c1ccc(N2CCCC2)cc1]

In a class of this embodiment, R² is hydrogen. In a subclass of this class, R³ is hydrogen. In another subclass of this class, R³ is methyl.

In another class of this embodiment, R² is methyl. In a subclass of this class, R³ is hydrogen. In another subclass of this class, R³ is methyl.

In one embodiment of the present invention, the microbial infection is a bacterial or fungal infection.

In one embodiment of the present invention, the microbial infection is a bacterial infection.

In one embodiment of the present invention, the microbial infection is a fungal infection.

In another embodiment of the present invention, the bacterial infection is caused by a drug-resistant bacterium.

In another embodiment of the present invention, the bacterial infection is caused by a gram-negative bacterium. In yet another embodiment of the present invention, the bacterial infection is caused by a gram-positive bacterium.

In one embodiment of the present invention, the bacterial infection is caused by at least one gram-negative organism selected from the group consisting of Acinetobacter spp., Actinobacillus spp., Aeromonas spp., Alcaligenes spp., Bacteroides spp., Bartonella spp., Bordetella spp., Branhamella spp., Brucella spp., Burkholderia spp., Campylobacter spp., Citrobacter spp., Coxiella spp., Edwarsiella spp., Ehrlichia spp., Eikenella spp., Enterobacter spp., Escherichia spp., Flavobacterium spp., Francisella spp., Fusobacterium spp., Haemophilus spp., Haemophilus spp., Helicobacter spp., Kingella spp., Klebsiella spp., Legionella spp., Moraxella spp., Morganella spp., Neisseria spp., Pasteurella spp., Plesiomonas spp., Porphyromonas spp., Prevotella spp., Prevotella spp., Prevotella spp., Proteus spp., Providencia spp., Pseudomonas spp., Ricketsia spp., Salmonella spp., Serratia spp., Shigella spp., Stenotrophomonas spp., Streptobacillus spp., Vibrio spp. and Yersinia spp..

In another embodiment of the present invention, the bacterial infection is caused by at least one gram-negative organism selected from the group consisting of Acinetobacter baumannii, Pseudomonas aeruginosa, Escherichia coli, Neisseria gonorrhoeae and Chlamydia trachomatis.

In one embodiment of the present invention, the bacterial infection is caused by at least one gram-positive organism selected from the group consisting of Bacillus spp., Listeria spp., Staphylococcus spp., Enterococcus spp., Clostridium spp., Streptococcus spp., Actinomyces spp. and Mycobacterium spp..

In another embodiment of the present invention, the bacterial infection is caused by at least one gram-positive organism selected from the group consisting of Staphylococcus aureus, Streptococcus pyogenes, Streptococcus pneumoniae, Clostridium difficile, Enterococcus Faecalis, and Enterococcus faecium.

In yet one embodiment of the present invention, the bacterial infection is caused by Staphylococcus aureus.

In one embodiment of the present invention, the bacterial infection is selected from one or more of the following: urinary tract infection, a respiratory infection, a surgical wound infection, a central line infection, bacteremia, bronchitis, sinusitis, pneumonia, prostatitis, a skin or soft tissue infection, an intra-abdominal infection, or a bacterial infection of febrile neutropenic patients.

In one embodiment of the present invention, the method further comprises the administration of one or more compounds selected from the group consisting of an antibiotic, an anti-inflammatory agent, a matrix metalloprotease inhibitor, a lipoxygenase inhibitor, a cytokine antagonist, an immunosuppressant, an anti-cancer agent, an anti-viral agent, a cytokine, a growth factor, an immunomodulator, a prostaglandin, and an anti-vascular hyperproliferation compound, either as part of a multiple dosage form together with said compound or as a single dosage form. In multiple dosage form a compound of Table 1 is administered separately with an agent described above, and in a single dosage form, a compound of Table 1 is combined an agent described above is administered in a single composition.

Non-limiting examples of classes of antibiotics suitable for administration with the compounds of the present invention, and compositions thereof, include quinolones, beta-lactams, macrolides, glycopeptides, and lipopetides.

Non-limiting examples of specific antibiotics include alatrofloxacin, altrofloxacin, amdinocillin, amoxicillin, ampicillin, azithromycin, bacampicillin, besifloxacin, carbenicillin, ceadroxil, cefaclor, cefazolin, cefditoren, cefinir, cefixime, cefprozil, ceftibuten, cefuroxime axetil, cephapirin, chloramphenicol, chlortetracycline, ciprofloxacin, cloxacillin, clarithromycin, clavulanate potassium, clindamycin phosphate, cloxacillin, cyclacillin, dactinomycin, daptomycin, dicloxacillin, dirithromycin, doxycycline, enoxacin, erythromycin, fosfomycin tromethamine, fluorometholone, gatifloxacin, gemifloxacin, gentamicin, grepafloxacin, hetacillin, kanamycin, levofloxacin, lincomycin, linezolid, lomefloxacin, maxaquin, mezlocillin, minocycline, moxifloxacin, mupirocin, nafcillin, netilmicin, norfloxacin, ofloxacin, oxacillin, oxytetracycline, penicillamine, penicillin G, penicillin V, piperacillin, plicamycin, rifamycin, rifabutin, rifampin, rifapentine, rifaximin, soarfloxacin, sparfloxacin, sulfamethoxazole, sulfisoxazole acetyl, telithromycin, ticarcillin, tobramycin, trimethoprim, troleandomycin, trovafloxacin, vancomycin, viomycin, and mixtures thereof.

In another embodiment, the method further comprises the step of administering to said patient an agent that increases the susceptibility of bacterial organisms to antibiotics.

Agents that increase the susceptibility of bacteria to antibiotics are known. A number of patents such as U.S. Pat. No. 5,523,288, U.S. Pat. No. 5,783,561 and U.S. Pat. No. 6,140,306 describe permeability-increasing bactericidal proteins that increase the bacterial susceptibility to antibiotics. Other agents that increase the susceptibility of bacteria to antibiotics have been described in the literature. (Vara, Microbiological Reviews, 56, 395-411 (1992); Tsubery, et al., J. Med. Chem. 43, 3085-3092 (2000)).

As used above, and throughout this disclosure, the following terms, unless otherwise indicated, shall be understood to have the following meanings:

“At least one” compound of Table 1 means 1, 2, 3 or 4 different compounds, but preferably one compound of form Table 1 is used in the claimed methods. Similarly, when “at least one” is used in connection with the additional agents used in the combinations, 1, 2, 3 or 4 additional agents are contemplated, but preferably one or two, more preferably one additional agent is used.

“Patient” includes both human and animals. A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

“PG” means protecting group.

“Mammal” means humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) , in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of defined by Table 1 or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Table 1 or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)-aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as 13-dimethylaminoethyl), carbamoyl-(C₁-C₂)alkyl, N,N-di (C₁-C₂)alkylearbamoyl-(C1-C2)alkyl and piperidino-, pyrrolidino- or morpholino(C₂-C₃)alkyl, and the like. Similarly, if a compound of Table 1 contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each oc-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Table 1 incorporates an amine functional group, a prodrug can be formed by the replacement of a hydrogen atom in the amine group with a group such as, for example, R-carbonyl, RO-carbonyl, NRR'-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N-(C₁-C₆)alkylaminoalkyl, —C(Y⁴)Y⁵ wherein Y⁴ is H or methyl and Y⁵ is mono-N— or di-N,N-(C₁-C₆)alkylamino morpholine, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example 1. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Table 1 can form salts which are also within the scope of this invention. Reference to a compound of Table 1 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Table 1 contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Table 1 may be formed, for example, by reacting a compound of Table 1 with an amount of acid or base, such as an equivalent amount, in a medium such as one in which the. salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Table 1, and salts, solvates, esters and prodrugs thereof, may exist in their tautomeric form (for example, as an amide or imino ether). All such tautomeric forms are contemplated herein as part of the present invention.

The compounds of Table 1 may contain asymmetric or chiral centers, and, therefore, exist in different stereoisomeric forms. It is intended that all stereoisomeric forms of the compounds of Table 1 as well as mixtures thereof, including racemic mixtures, four' part of the present invention. In addition, the present invention embraces all geometric and positional isomers. For example, if a compound of Table 1 incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Masher's acid chloride), separating the diastereomers and converting (e.g., hydrolyzing) the individual diastereomers to the corresponding pure enantiomers. Also, some of the compounds of Table 1 may be atropisomers (e.g., substituted biaryls) and are considered as part of this invention.

Enantiomers can also be separated by use of chiral HPLC column.

It is also possible that the compounds of Table 1 may exist in different tautomeric forms, and all such forms are embraced within the scope of the invention. Also, for example, all keto-enol and imine-enamine forms of the compounds are included in the invention. All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Table 1 incorporates a double bond or a fused ring, both the cis- and trans-forms, as well as mixtures, are embraced within the scope of the invention. Also, for example, all keto-enol and imine-enamine forms of the compounds are included in the invention.). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Table I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Table I can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Table 1, in particular those containing isotopes with longer half lives (T1/2 >1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Table 1, and of the salts, solvates, esters and prodrugs of the compounds of Table 1, are intended to be included in the present invention.

Those skilled in the art will appreciate that for some of the compounds of Table 1, one isomer will show greater pharmacological activity than other isomers.

One to three compounds of Table 1 can be administered in the methods of the invention, preferably one.

Preferably the compound of Table 1 is administered orally.

The compounds listed above can be administered to an animal orally, intravenously, by inhalation (e.g., to treat fungal infections in the lungs) or topically (e.g. to treat microbial infections of the skin or mucous membranes). Preferably the compound(s) of the invention listed above is administered orally or intravenously, more preferably orally.

For preparing pharmaceutical compositions from the compounds useful in the method of this invention, inert, pharmaceutically acceptable carriers can be either solid or liquid. Solid form preparations include powders, tablets, dispersible granules, capsules, cachets and suppositories. The powders and tablets may be comprised of from about 0.1 to about 99 percent active ingredient. Suitable solid carriers are known in the art, e.g. magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration. Aerosol preparations suitable for inhalation may include solutions and solids in powder form, which may be in combination with a pharmaceutically acceptable carrier, such as an inert compressed gas. Also included are solid form preparations which are intended to be converted, shortly before use, to liquid form preparations for either oral or parenteral administration. Such liquid forms include solutions, suspensions and emulsions.

The compounds useful in the method of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in unit dosage form. In such form, the preparation is subdivided into unit doses containing appropriate quantities of the active component, e.g., an effective amount to achieve the desired purpose.

The quantity of compound listed above in a unit dose of preparation may be varied or adjusted from about 0.1 mg to 1000 mg, more preferably from about 1 mg to 300 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day if desired.

The amount and frequency of administration of the compounds of Table 1 will be regulated according to the judgment of the attending clinician considering such factors as age, condition and size of the patient as well as severity of the symptoms being treated. A typical recommended dosage regimen for compounds of Table 1 is oral administration of from 10 mg to 2000 mg/day preferably 10 to 1000 mg/day, in two to four divided doses to provide relief from the diseases or conditions listed above.

The doses and dosage regimen of the other agents used in the treatment of diseases or conditions listed above will be determined by the attending clinician in view of the approved doses and dosage regimen in the package insert, taking into consideration the age, sex and condition of the patient and the severity of the disease. When administered in combination, the compound(s) of Table 1 and the other agent(s) for treating diseases or conditions listed above can be administered simultaneously or sequentially. This is particularly useful when the components of the combination are preferably given on different dosing schedules, e.g., one component is administered once daily and another every six hours, or when the preferred pharmaceutical compositions are different, e.g. one is preferably a tablet and one is a capsule. A kit comprising the separate dosage forms is therefore advantageous.

When the invention comprises a combination of one or more compounds listed above and one or more other antifungal agents, the active components may be co-administered simultaneously or sequentially, or a single pharmaceutical composition comprising one or more compounds listed above and one or more other antifungal agents in a pharmaceutically acceptable carrier can be administered. The components of the combination can be administered individually or together in any conventional dosage form such as capsule, tablet, powder, cachet, suspension, solution, suppository, nasal spray, etc. The dosages of the other antifungal agents can be determined from published material, and may range from 1 to 1000 mg per dose. When used in combination, the dosage levels of the individual components are preferably lower than the recommended individual dosages because of the advantageous effect of the combination.

The compounds of the invention can be made according to the processes described below. The compounds of this invention are also exemplified in the examples below, which examples should not be construed as limiting the scope of the disclosure. Alternative mechanistic pathways and analogous structures within the scope of the invention may be apparent to those skilled in the art.

General Methods The general methods described in this paragraph were used unless stated otherwise in the examples below. All solvents and reagents were used as received. Proton NMR spectra were obtained using a Varian XL-400 (400 MHz) instrument and were reported as parts per million (ppm) downfield from Me₄Si. LCMS analysis was performed using an Applied Biosystems API-100 mass spectrometer equipped with a Shimadzu SCL-10A LC column: Altech platinum C18, 3 um,33 mm×7 nun ID; gradient flow: 0 min, 10% CH₃CN; 5 min, 95% CH₃CN; 7 min, 95% CH₃CN; 7.5 min, 10% CH₃CN; 9 min, stop. Flash column chromatography was performed using Selecto Scientiic flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

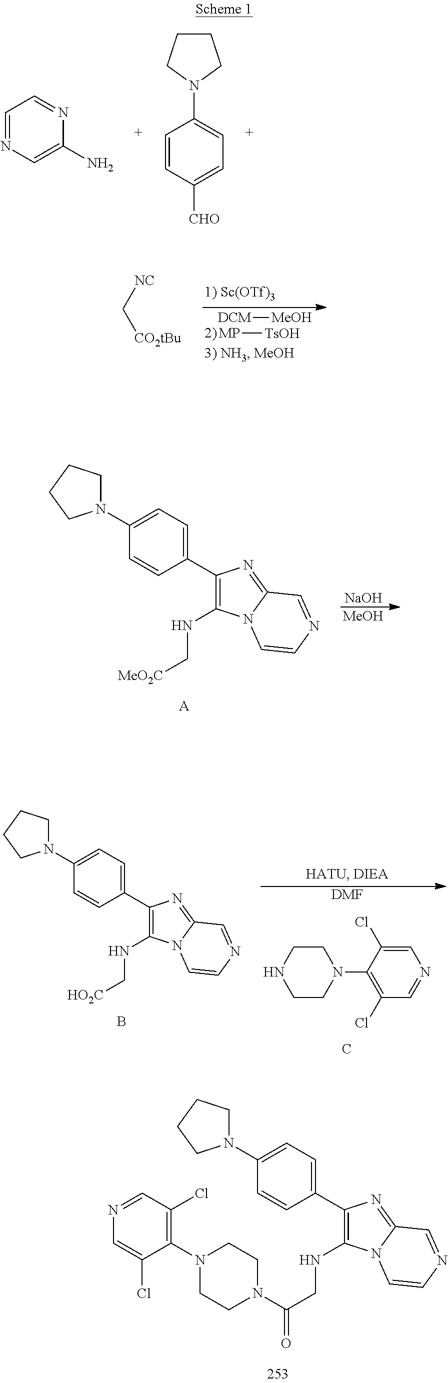

To a DCM-MeOH (3:1) solution (21 mL) of aminopyrazine (1.00 g, 10.52 mmol), 4-(1-pyrrolidino)benzaldehyde (2.11 g, 12.0 mmol) and scandium(III) trifluoromethanesulfonate (259 mg, 0.50 mmol) at room temperature was added Cert-butylisocyanoacetate (1.85 mL, 12.7 mmol). The reaction mixture was stirred at room temperature for 36 hrs. The solution was treated with MP-TsOH resin (4.13 mmol/g, 12.8 g, 52.9 mmol) and stirred at room temperature for 3 days. The resin was filtered, washed with DCM (3×), MeOH (3×) and dried in vacuo. The resin was treated with 2N NH₃-MeOH (50 mL) at room temperature for 1 h. The solvent was filtered off and the resin was washed with both DCM and MeOH. The combined filtrate was concentrated in vacuo and dried in vacuo to afford the desired methyl ester A (2.65 g 7.76 mmol).

The methyl ester A (2.65 g, 7.76 mmol) was dissolved in a MeOH-THF (4:1) solution (100 mL) and 1N sodium hydroxide solution (9.0 mL, 9.0 mmol) was added. The reaction mixture was stirred at room temperature overnight. The solvent was evaporated in vacua to afford the desired acid 13 (50.2 mg, 0.112 mmol).

To a DMF (2 mL) solution of the acid B (50.2 mg, 0.112 mmol) and amine C (31.3 mg, 0.135 mmol), at room temperature under nitrogen, was added diisopropylethylamine (0.2 mL, 1.15 mmol). Let stir for 5 minutes and HATU (64 mg, 0.168 mmol) was added. After stirring at room temperature for 3 hours, ethyl acetate and IN NaOH were added. The two layers were separated, the aqueous layer was back extracted with ethyl acetate (2×). The organic layers were combined, washed with water (3×), dried over Na₂SO₄ and concentrated in vacuo. The crude product was purified by flash chromatography [0-4%, (2N NH₃-MeOH)-DCM] to afford the desired amide 253 (60.6 mg) as an orange-brown solid.

Using procedures analogous to those described above, the compounds of Table 1 were synthesized.

TABLE 1

| I.D. | Structure | M + H |
|---|---|---|
| 1 | 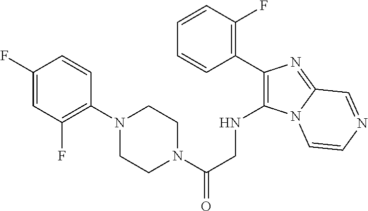 [SMILES: O=C(CNc1c(-c2ccccc2F)nc2cnccn12)N1CCN(c2ccc(F)cc2F)CC1] | — |
| 2 | 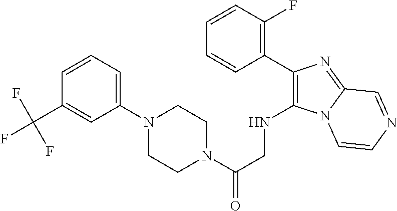 [SMILES: O=C(CNc1c(-c2ccccc2F)nc2cnccn12)N1CCN(c2cccc(C(F)(F)F)c2)CC1] | — |
| 3 | 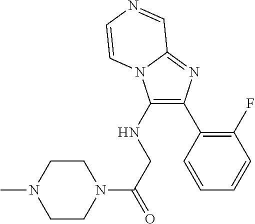 [SMILES: CN1CCN(C(=O)CNc2c(-c3ccccc3F)nc3cnccn23)CC1] | — |
| 4 | 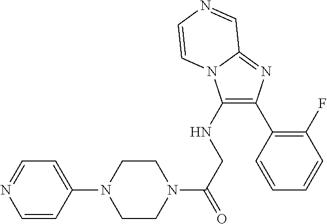 [SMILES: O=C(CNc1c(-c2ccccc2F)nc2cnccn12)N1CCN(c2ccncc2)CC1] | — |
| 5 | 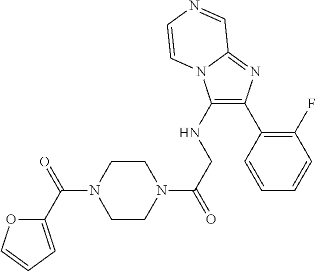 [SMILES: O=C(CNc1c(-c2ccccc2F)nc2cnccn12)N1CCN(C(=O)c2ccco2)CC1] | — |
| 6 | 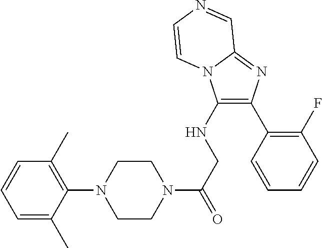 [SMILES: Cc1cccc(C)c1N1CCN(C(=O)CNc2c(-c3ccccc3F)nc3cnccn23)CC1] | — |
| 7 | 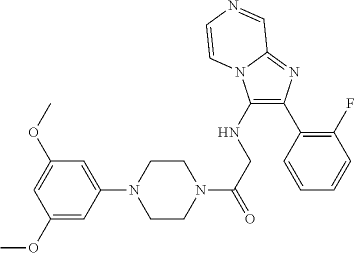 [SMILES: COc1cc(OC)cc(N2CCN(C(=O)CNc3c(-c4ccccc4F)nc4cnccn34)CC2)c1] | — |
| 8 | 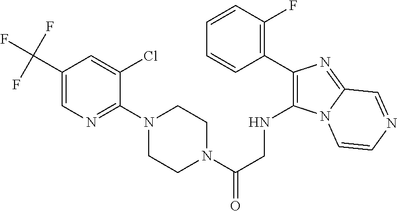 [SMILES: O=C(CNc1c(-c2ccccc2F)nc2cnccn12)N1CCN(c2ncc(C(F)(F)F)cc2Cl)CC1] | — |
| 9 | 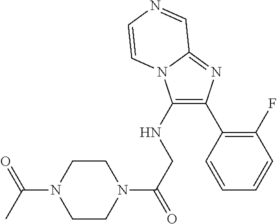 [SMILES: CC(=O)N1CCN(C(=O)CNc2c(-c3ccccc3F)nc3cnccn23)CC1] | — |
| 10 | 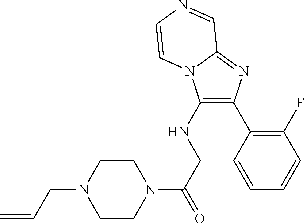 [SMILES: C=CCN1CCN(C(=O)CNc2c(-c3ccccc3F)nc3cnccn23)CC1] | — |
| 11 | 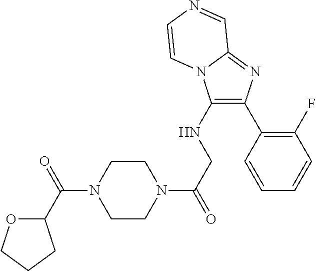 [SMILES: O=C(CNc1c(-c2ccccc2F)nc2cnccn12)N1CCN(C(=O)C2CCCO2)CC1] | — |
| 12 | 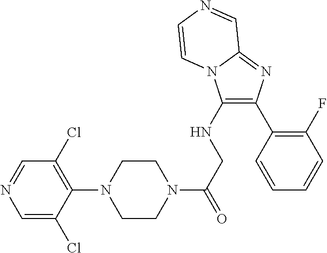 [SMILES: O=C(CNc1c(-c2ccccc2F)nc2cnccn12)N1CCN(c2c(Cl)cncc2Cl)CC1] | — |
| 13 | 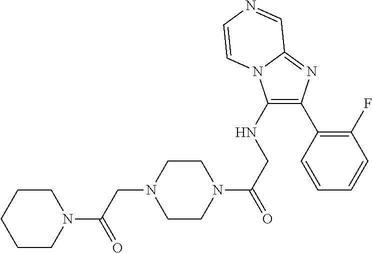 [SMILES: O=C(CNc1c(-c2ccccc2F)nc2cnccn12)N1CCN(CC(=O)N2CCCCC2)CC1] | — |
| 14 | 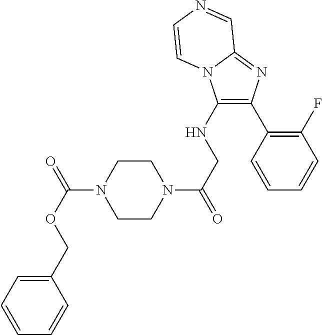 [SMILES: O=C(CNc1c(-c2ccccc2F)nc2cnccn12)N1CCN(C(=O)OCc2ccccc2)CC1] | — |
| 15 | 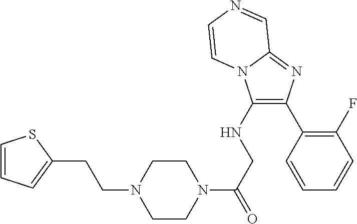 [SMILES: O=C(CNc1c(-c2ccccc2F)nc2cnccn12)N1CCN(CCc2cccs2)CC1] | — |
| 16 | 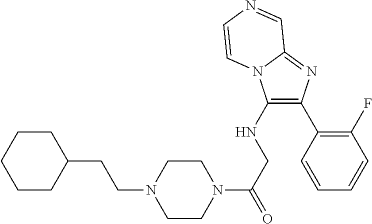 [SMILES: O=C(CNc1c(-c2ccccc2F)nc2cnccn12)N1CCN(CCC2CCCCC2)CC1] | — |
| 17 | 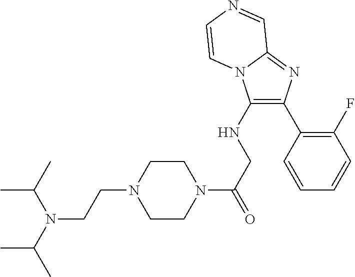 [SMILES: CC(C)N(CCN1CCN(C(=O)CNc2c(-c3ccccc3F)nc3cnccn23)CC1)C(C)C] | — |
| 18 | 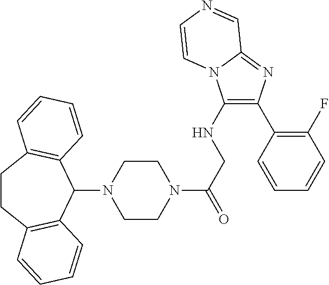 [SMILES: O=C(CNc1c(-c2ccccc2F)nc2cnccn12)N1CCN(C2c3ccccc3CCc3ccccc32)CC1] | — |
| 19 | 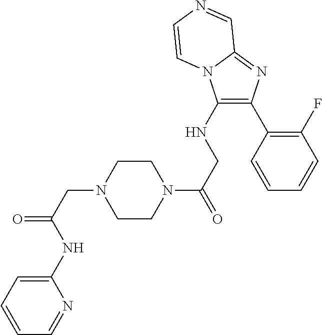 [SMILES: O=C(CN1CCN(C(=O)CNc2c(-c3ccccc3F)nc3cnccn23)CC1)Nc1ccccn1] | — |
| 20 | 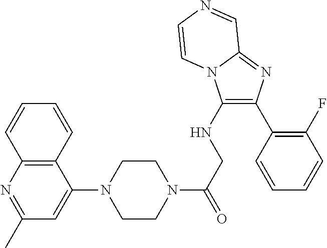 [SMILES: Cc1cc(N2CCN(C(=O)CNc3c(-c4ccccc4F)nc4cnccn34)CC2)c2ccccc2n1] | — |
| 21 | 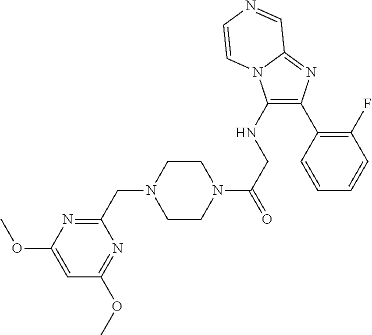 [SMILES: COc1cc(OC)nc(CN2CCN(C(=O)CNc3c(-c4ccccc4F)nc4cnccn34)CC2)n1] | — |
| 22 | 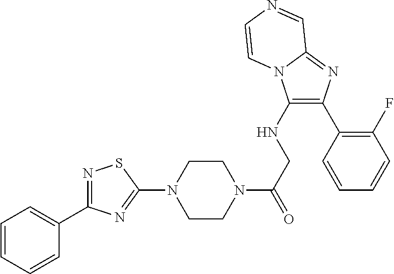 [SMILES: O=C(CNc1c(-c2ccccc2F)nc2cnccn12)N1CCN(c2nc(-c3ccccc3)ns2)CC1] | — |
| 23 | 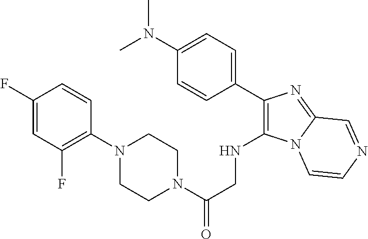 [SMILES: CN(C)c1ccc(-c2nc3cnccn3c2NCC(=O)N2CCN(c3ccc(F)cc3F)CC2)cc1] | — |
| 24 | 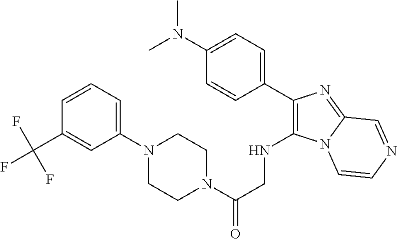 [SMILES: CN(C)c1ccc(-c2nc3cnccn3c2NCC(=O)N2CCN(c3cccc(C(F)(F)F)c3)CC2)cc1] | — |
| 25 | 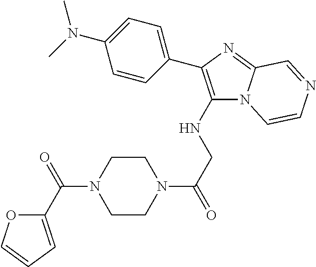 [SMILES: CN(C)c1ccc(-c2nc3cnccn3c2NCC(=O)N2CCN(C(=O)c3ccco3)CC2)cc1] | — |
| 26 | 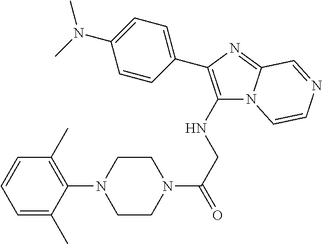 [SMILES: Cc1cccc(C)c1N1CCN(C(=O)CNc2c(-c3ccc(N(C)C)cc3)nc3cnccn23)CC1] | 484.3 |
| 27 | 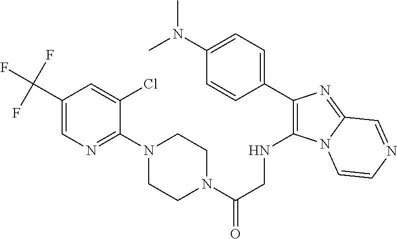 [SMILES: CN(C)c1ccc(-c2nc3cnccn3c2NCC(=O)N2CCN(c3ncc(C(F)(F)F)cc3Cl)CC2)cc1] | — |
| 28 | 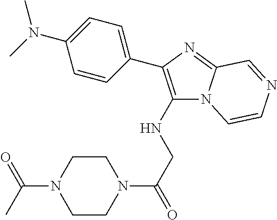 [SMILES: CC(=O)N1CCN(C(=O)CNc2c(-c3ccc(N(C)C)cc3)nc3cnccn23)CC1] | — |
| 29 | 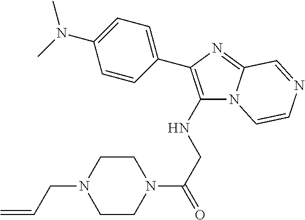 [SMILES: C=CCN1CCN(C(=O)CNc2c(-c3ccc(N(C)C)cc3)nc3cnccn23)CC1] | — |
| 30 | 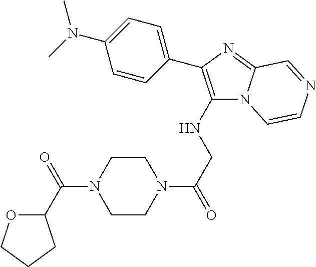 [SMILES: CN(C)c1ccc(-c2nc3cnccn3c2NCC(=O)N2CCN(C(=O)C3CCCO3)CC2)cc1] | — |
| 31 | 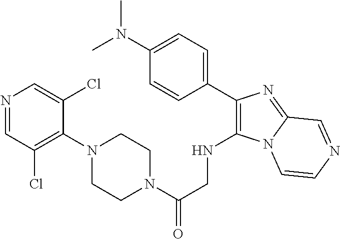 [SMILES: CN(C)c1ccc(-c2nc3cnccn3c2NCC(=O)N2CCN(c3c(Cl)cncc3Cl)CC2)cc1] | — |
| 32 | 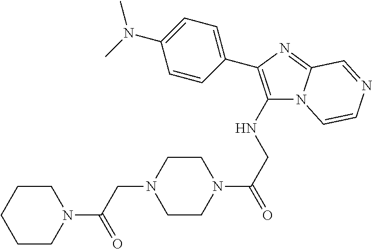 [SMILES: CN(C)c1ccc(-c2nc3cnccn3c2NCC(=O)N2CCN(CC(=O)N3CCCCC3)CC2)cc1] | — |
| 33 | 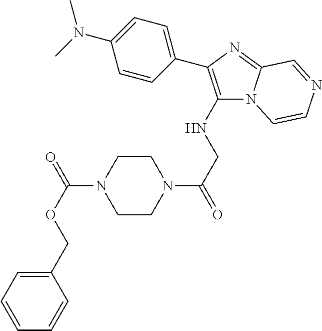 [SMILES: CN(C)c1ccc(-c2nc3cnccn3c2NCC(=O)N2CCN(C(=O)OCc3ccccc3)CC2)cc1] | — |
| 34 | 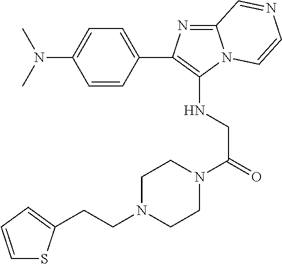 [SMILES: CN(C)c1ccc(-c2nc3cnccn3c2NCC(=O)N2CCN(CCc3cccs3)CC2)cc1] | — |
| 35 | 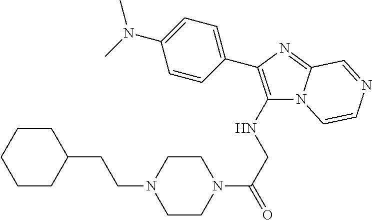 [SMILES: CN(C)c1ccc(-c2nc3cnccn3c2NCC(=O)N2CCN(CCC3CCCCC3)CC2)cc1] | — |
| 36 | 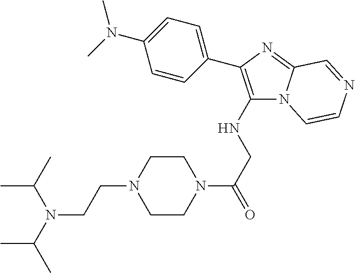 [SMILES: CC(C)N(CCN1CCN(C(=O)CNc2c(-c3ccc(N(C)C)cc3)nc3cnccn23)CC1)C(C)C] | — |
| 37 | 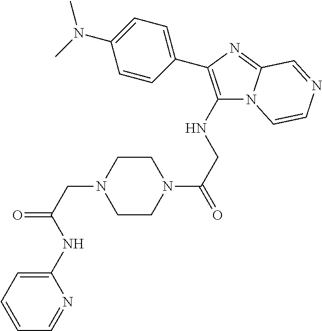 [SMILES: CN(C)c1ccc(-c2nc3cnccn3c2NCC(=O)N2CCN(CC(=O)Nc3ccccn3)CC2)cc1] | — |
| 38 | 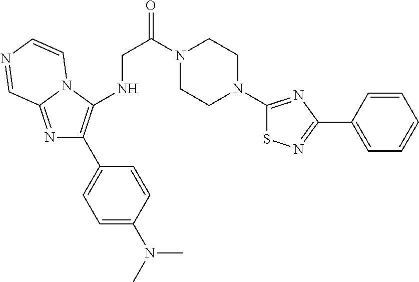 [SMILES: CN(C)c1ccc(-c2nc3cnccn3c2NCC(=O)N2CCN(c3nc(-c4ccccc4)ns3)CC2)cc1] | — |
| 39 | 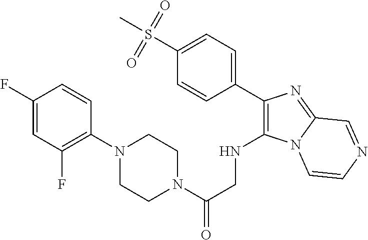 [SMILES: CS(=O)(=O)c1ccc(-c2nc3cnccn3c2NCC(=O)N2CCN(c3ccc(F)cc3F)CC2)cc1] | — |
| 40 | 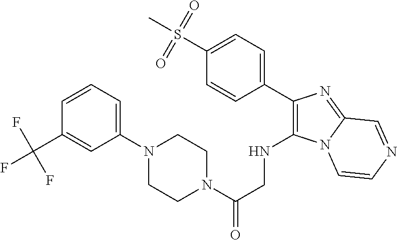 [SMILES: CS(=O)(=O)c1ccc(-c2nc3cnccn3c2NCC(=O)N2CCN(c3cccc(C(F)(F)F)c3)CC2)cc1] | — |
| 41 | 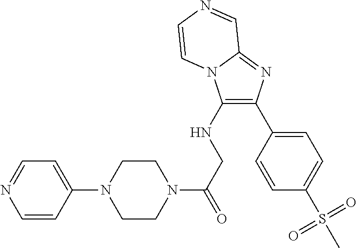 [SMILES: CS(=O)(=O)c1ccc(-c2nc3cnccn3c2NCC(=O)N2CCN(c3ccncc3)CC2)cc1] | — |
| 42 | 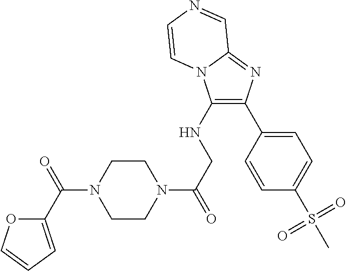 [SMILES: CS(=O)(=O)c1ccc(-c2nc3cnccn3c2NCC(=O)N2CCN(C(=O)c3ccco3)CC2)cc1] | — |
| 43 | 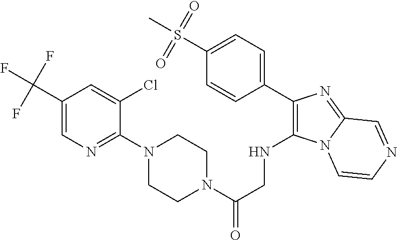 [SMILES: CS(=O)(=O)c1ccc(-c2nc3cnccn3c2NCC(=O)N2CCN(c3ncc(C(F)(F)F)cc3Cl)CC2)cc1] | — |
| 44 | 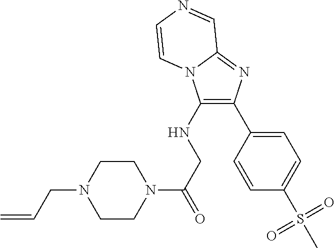 [SMILES: C=CCN1CCN(C(=O)CNc2c(-c3ccc(S(C)(=O)=O)cc3)nc3cnccn23)CC1] | — |
| 45 | 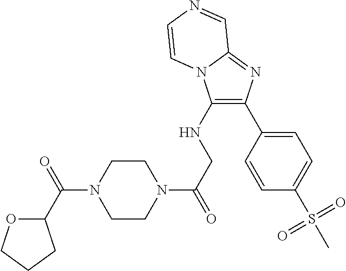 [SMILES: CS(=O)(=O)c1ccc(-c2nc3cnccn3c2NCC(=O)N2CCN(C(=O)C3CCCO3)CC2)cc1] | — |
| 46 | 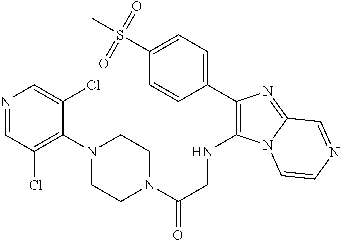 [SMILES: CS(=O)(=O)c1ccc(-c2nc3cnccn3c2NCC(=O)N2CCN(c3c(Cl)cncc3Cl)CC2)cc1] | — |
| 47 | 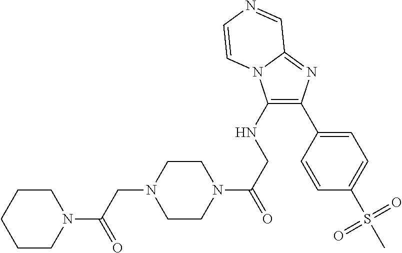 [SMILES: CS(=O)(=O)c1ccc(-c2nc3cnccn3c2NCC(=O)N2CCN(CC(=O)N3CCCCC3)CC2)cc1] | — |
| 48 | 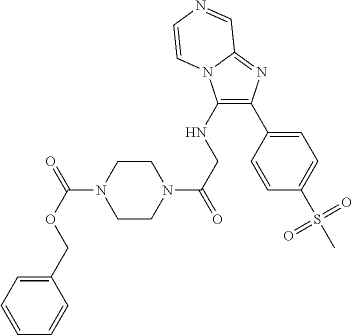 [SMILES: CS(=O)(=O)c1ccc(-c2nc3cnccn3c2NCC(=O)N2CCN(C(=O)OCc3ccccc3)CC2)cc1] | — |
| 49 | 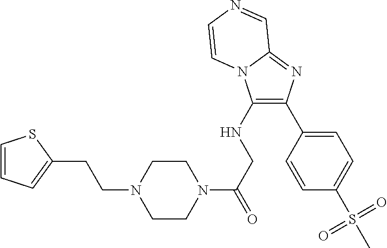 [SMILES: CS(=O)(=O)c1ccc(-c2nc3cnccn3c2NCC(=O)N2CCN(CCc3cccs3)CC2)cc1] | — |
| 50 | 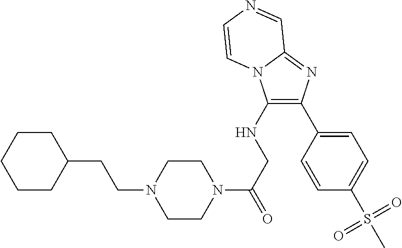 [SMILES: CS(=O)(=O)c1ccc(-c2nc3cnccn3c2NCC(=O)N2CCN(CCC3CCCCC3)CC2)cc1] | — |
| 51 | 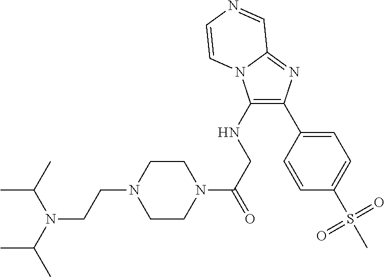 [SMILES: CC(C)N(CCN1CCN(C(=O)CNc2c(-c3ccc(S(C)(=O)=O)cc3)nc3cnccn23)CC1)C(C)C] | — |
| 52 | 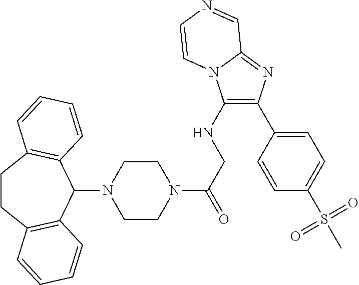 [SMILES: CS(=O)(=O)c1ccc(-c2nc3cnccn3c2NCC(=O)N2CCN(C3c4ccccc4CCc4ccccc43)CC2)cc1] | — |
| 53 | 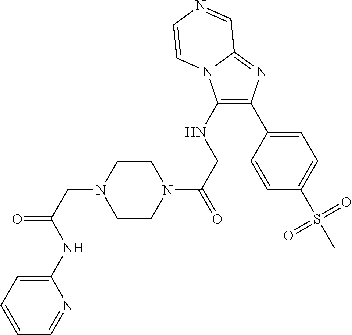 [SMILES: CS(=O)(=O)c1ccc(-c2nc3cnccn3c2NCC(=O)N2CCN(CC(=O)Nc3ccccn3)CC2)cc1] | — |
| 54 | 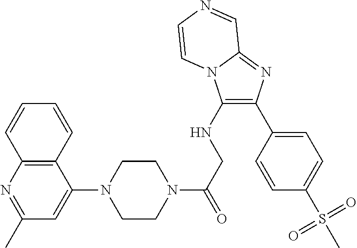 [SMILES: Cc1cc(N2CCN(C(=O)CNc3c(-c4ccc(S(C)(=O)=O)cc4)nc4cnccn34)CC2)c2ccccc2n1] | — |
| 55 | 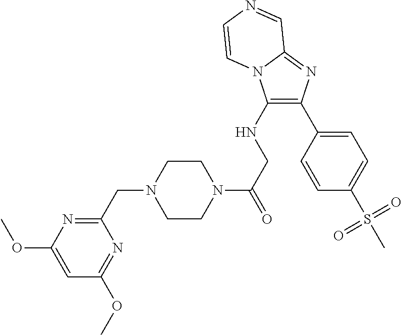 [SMILES: COc1cc(OC)nc(CN2CCN(C(=O)CNc3c(-c4ccc(S(C)(=O)=O)cc4)nc4cnccn34)CC2)n1] | — |
| 56 | 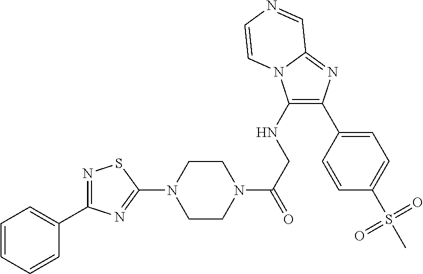 [SMILES: CS(=O)(=O)c1ccc(-c2nc3cnccn3c2NCC(=O)N2CCN(c3nc(-c4ccccc4)ns3)CC2)cc1] | — |
| 57 | 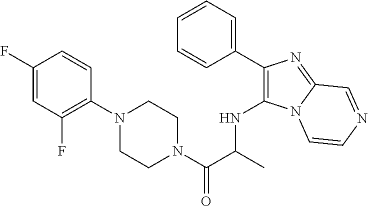 [SMILES: CC(Nc1c(-c2ccccc2)nc2cnccn12)C(=O)N1CCN(c2ccc(F)cc2F)CC1] | — |
| 58 | 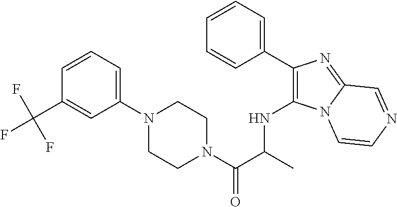 [SMILES: CC(Nc1c(-c2ccccc2)nc2cnccn12)C(=O)N1CCN(c2cccc(C(F)(F)F)c2)CC1] | — |
| 59 | 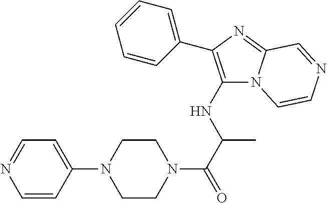 [SMILES: CC(Nc1c(-c2ccccc2)nc2cnccn12)C(=O)N1CCN(c2ccncc2)CC1] | — |
| 60 | 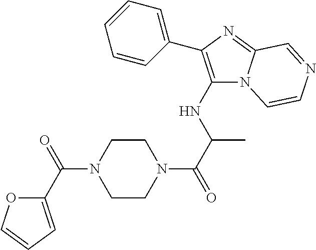 [SMILES: CC(Nc1c(-c2ccccc2)nc2cnccn12)C(=O)N1CCN(C(=O)c2ccco2)CC1] | — |
| 61 | 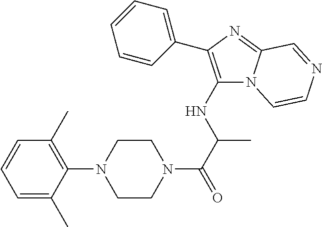 [SMILES: Cc1cccc(C)c1N1CCN(C(=O)C(C)Nc2c(-c3ccccc3)nc3cnccn23)CC1] | — |
| 62 | 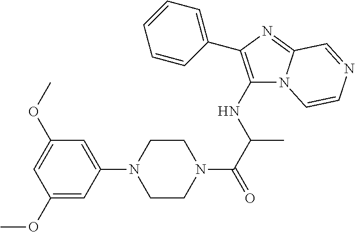 [SMILES: COc1cc(OC)cc(N2CCN(C(=O)C(C)Nc3c(-c4ccccc4)nc4cnccn34)CC2)c1] | — |
| 63 | 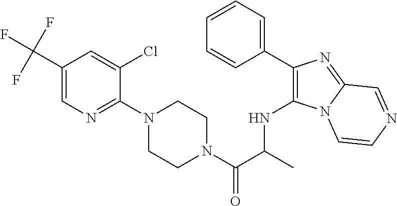 [SMILES: CC(Nc1c(-c2ccccc2)nc2cnccn12)C(=O)N1CCN(c2ncc(C(F)(F)F)cc2Cl)CC1] | — |
| 64 | 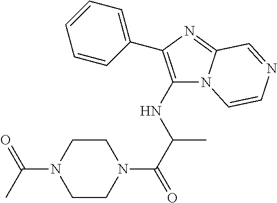 [SMILES: CC(=O)N1CCN(C(=O)C(C)Nc2c(-c3ccccc3)nc3cnccn23)CC1] | — |
| 65 | 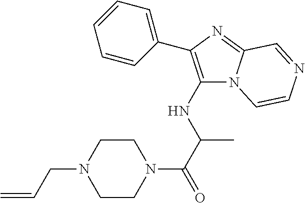 [SMILES: C=CCN1CCN(C(=O)C(C)Nc2c(-c3ccccc3)nc3cnccn23)CC1] | — |
| 66 | 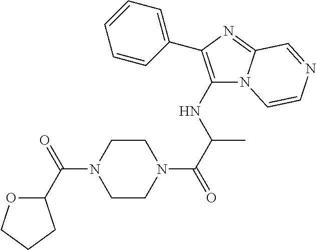 [SMILES: CC(Nc1c(-c2ccccc2)nc2cnccn12)C(=O)N1CCN(C(=O)C2CCCO2)CC1] | — |
| 67 | 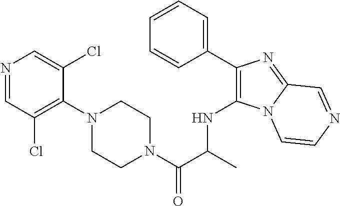 [SMILES: CC(Nc1c(-c2ccccc2)nc2cnccn12)C(=O)N1CCN(c2c(Cl)cncc2Cl)CC1] | — |
| 68 | 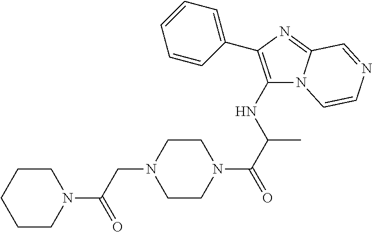 [SMILES: CC(Nc1c(-c2ccccc2)nc2cnccn12)C(=O)N1CCN(CC(=O)N2CCCCC2)CC1] | — |
| 69 | 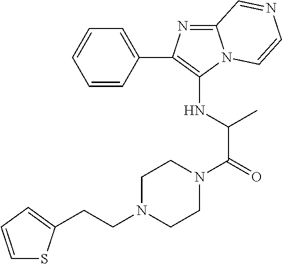 [SMILES: CC(Nc1c(-c2ccccc2)nc2cnccn12)C(=O)N1CCN(CCc2cccs2)CC1] | — |
| 70 | 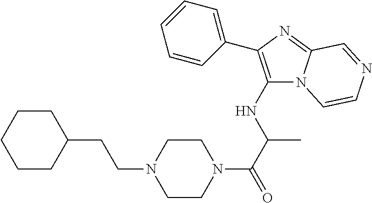 [SMILES: CC(Nc1c(-c2ccccc2)nc2cnccn12)C(=O)N1CCN(CCC2CCCCC2)CC1] | — |
| 71 | 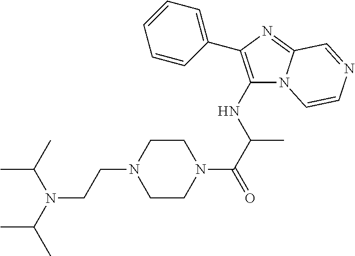 [SMILES: CC(Nc1c(-c2ccccc2)nc2cnccn12)C(=O)N1CCN(CCN(C(C)C)C(C)C)CC1] | — |
| 72 | 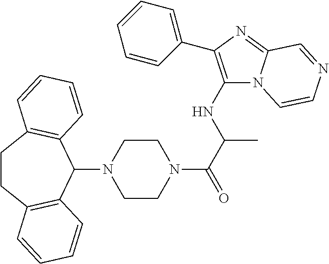 [SMILES: CC(Nc1c(-c2ccccc2)nc2cnccn12)C(=O)N1CCN(C2c3ccccc3CCc3ccccc32)CC1] | — |
| 73 | 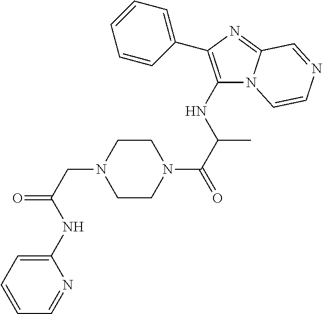 [SMILES: CC(Nc1c(-c2ccccc2)nc2cnccn12)C(=O)N1CCN(CC(=O)Nc2ccccn2)CC1] | — |
| 74 | 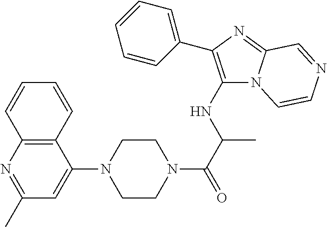 [SMILES: Cc1cc(N2CCN(C(=O)C(C)Nc3c(-c4ccccc4)nc4cnccn34)CC2)c2ccccc2n1] | — |
| 75 | 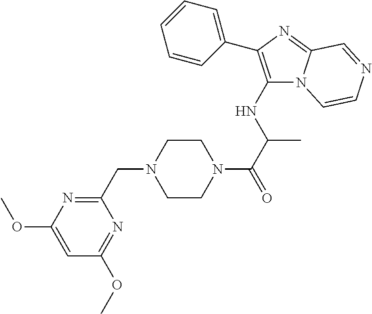 [SMILES: COc1cc(OC)nc(CN2CCN(C(=O)C(C)Nc3c(-c4ccccc4)nc4cnccn34)CC2)n1] | — |
| 76 | 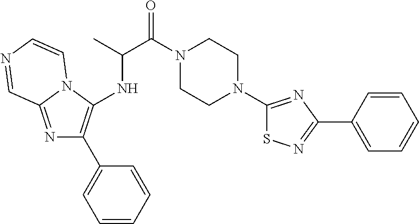 [SMILES: CC(Nc1c(-c2ccccc2)nc2cnccn12)C(=O)N1CCN(c2nc(-c3ccccc3)ns2)CC1] | — |
| 77 | 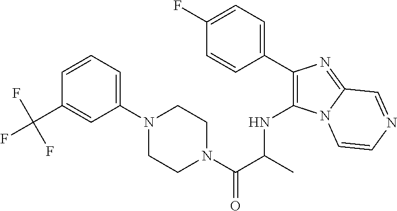 [SMILES: CC(Nc1c(-c2ccc(F)cc2)nc2cnccn12)C(=O)N1CCN(c2cccc(C(F)(F)F)c2)CC1] | — |
| 78 | 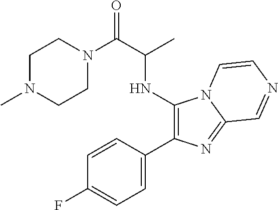 [SMILES: CC(Nc1c(-c2ccc(F)cc2)nc2cnccn12)C(=O)N1CCN(C)CC1] | — |
| 79 | 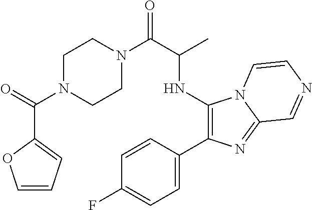 [SMILES: CC(Nc1c(-c2ccc(F)cc2)nc2cnccn12)C(=O)N1CCN(C(=O)c2ccco2)CC1] | — |
| 80 | 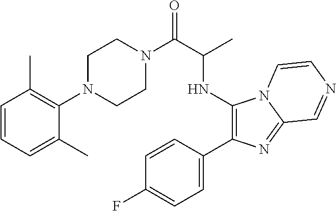 [SMILES: Cc1cccc(C)c1N1CCN(C(=O)C(C)Nc2c(-c3ccc(F)cc3)nc3cnccn23)CC1] | — |
| 81 | 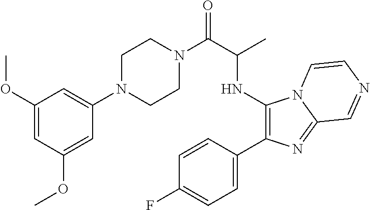 [SMILES: COc1cc(OC)cc(N2CCN(C(=O)C(C)Nc3c(-c4ccc(F)cc4)nc4cnccn34)CC2)c1] | — |
| 82 | 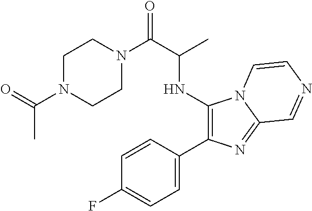 [SMILES: CC(=O)N1CCN(C(=O)C(C)Nc2c(-c3ccc(F)cc3)nc3cnccn23)CC1] | — |
| 83 | 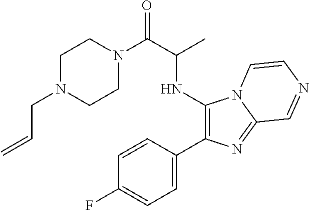 [SMILES: C=CCN1CCN(C(=O)C(C)Nc2c(-c3ccc(F)cc3)nc3cnccn23)CC1] | — |
| 84 | 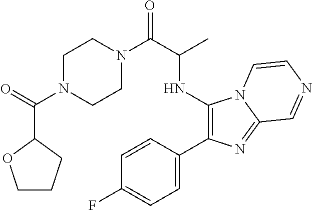 [SMILES: CC(Nc1c(-c2ccc(F)cc2)nc2cnccn12)C(=O)N1CCN(C(=O)C2CCCO2)CC1] | — |
| 85 | 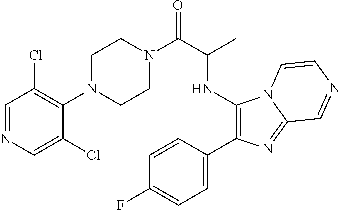 [SMILES: CC(Nc1c(-c2ccc(F)cc2)nc2cnccn12)C(=O)N1CCN(c2c(Cl)cncc2Cl)CC1] | — |
| 86 | 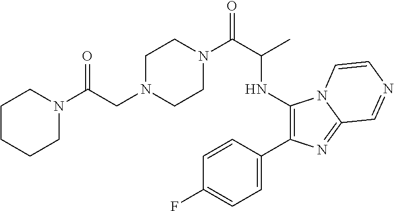 [SMILES: CC(Nc1c(-c2ccc(F)cc2)nc2cnccn12)C(=O)N1CCN(CC(=O)N2CCCCC2)CC1] | — |
| 87 | 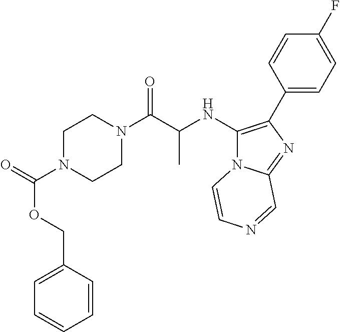 [SMILES: CC(Nc1c(-c2ccc(F)cc2)nc2cnccn12)C(=O)N1CCN(C(=O)OCc2ccccc2)CC1] | — |
| 88 | 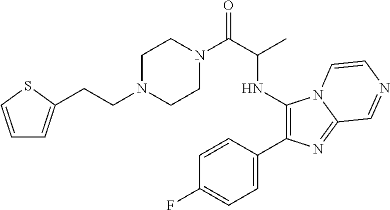 [SMILES: CC(Nc1c(-c2ccc(F)cc2)nc2cnccn12)C(=O)N1CCN(CCc2cccs2)CC1] | — |
| 89 | 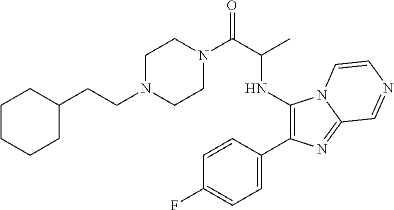 [SMILES: CC(Nc1c(-c2ccc(F)cc2)nc2cnccn12)C(=O)N1CCN(CCC2CCCCC2)CC1] | — |
| 90 | 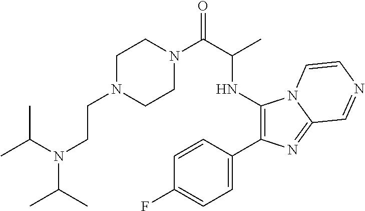 [SMILES: CC(Nc1c(-c2ccc(F)cc2)nc2cnccn12)C(=O)N1CCN(CCN(C(C)C)C(C)C)CC1] | — |
| 91 | 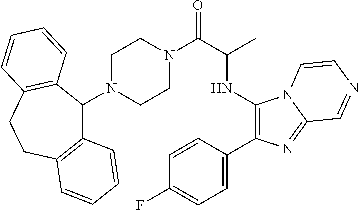 [SMILES: CC(Nc1c(-c2ccc(F)cc2)nc2cnccn12)C(=O)N1CCN(C2c3ccccc3CCc3ccccc32)CC1] | — |
| 92 | 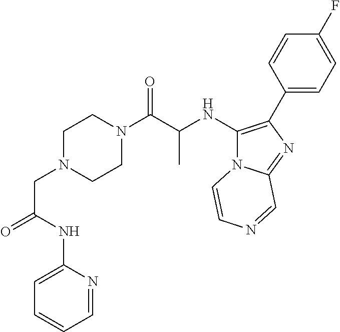 [SMILES: CC(Nc1c(-c2ccc(F)cc2)nc2cnccn12)C(=O)N1CCN(CC(=O)Nc2ccccn2)CC1] | — |
| 93 | 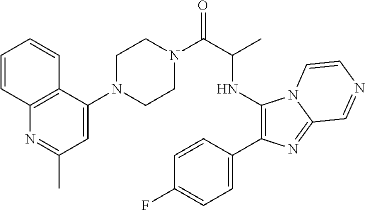 [SMILES: Cc1cc(N2CCN(C(=O)C(C)Nc3c(-c4ccc(F)cc4)nc4cnccn34)CC2)c2ccccc2n1] | — |
| 94 | 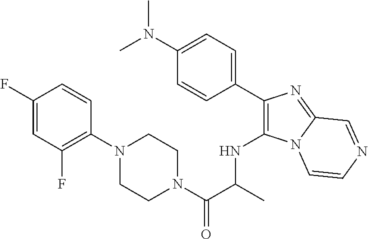 [SMILES: CC(Nc1c(-c2ccc(N(C)C)cc2)nc2cnccn12)C(=O)N1CCN(c2ccc(F)cc2F)CC1] | — |
| 95 | 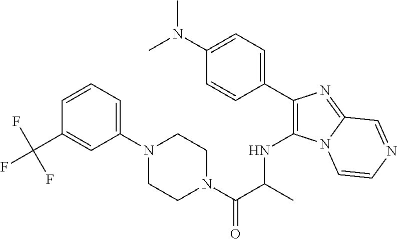 [SMILES: CC(Nc1c(-c2ccc(N(C)C)cc2)nc2cnccn12)C(=O)N1CCN(c2cccc(C(F)(F)F)c2)CC1] | — |
| 96 | 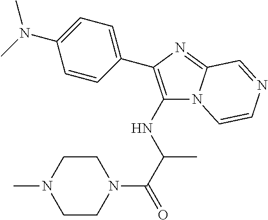 [SMILES: CC(Nc1c(-c2ccc(N(C)C)cc2)nc2cnccn12)C(=O)N1CCN(C)CC1] | — |
| 97 | 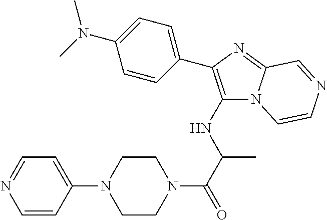 [SMILES: CC(Nc1c(-c2ccc(N(C)C)cc2)nc2cnccn12)C(=O)N1CCN(c2ccncc2)CC1] | — |
| 98 | 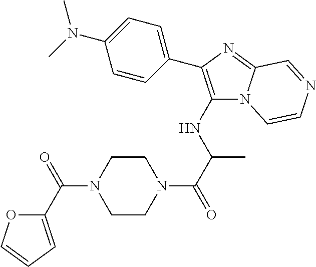 [SMILES: CC(Nc1c(-c2ccc(N(C)C)cc2)nc2cnccn12)C(=O)N1CCN(C(=O)c2ccco2)CC1] | — |
| 99 | 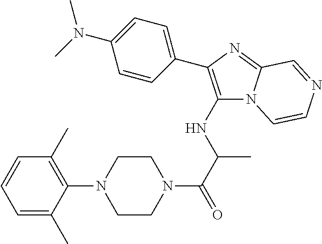 [SMILES: Cc1cccc(C)c1N1CCN(C(=O)C(C)Nc2c(-c3ccc(N(C)C)cc3)nc3cnccn23)CC1] | — |
| 100 | 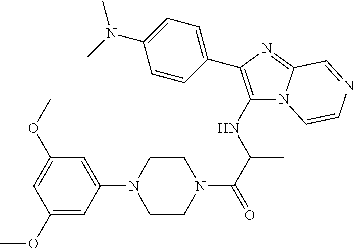 [SMILES: COc1cc(OC)cc(N2CCN(C(=O)C(C)Nc3c(-c4ccc(N(C)C)cc4)nc4cnccn34)CC2)c1] | — |
| 101 | 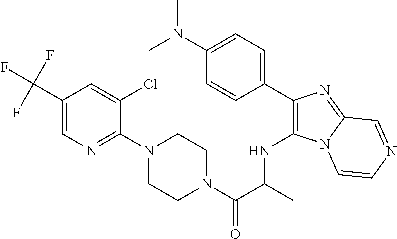 [SMILES: CC(Nc1c(-c2ccc(N(C)C)cc2)nc2cnccn12)C(=O)N1CCN(c2ncc(C(F)(F)F)cc2Cl)CC1] | — |
| 102 | 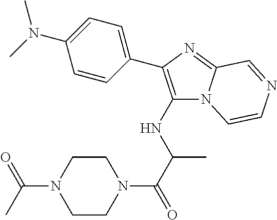 [SMILES: CC(=O)N1CCN(C(=O)C(C)Nc2c(-c3ccc(N(C)C)cc3)nc3cnccn23)CC1] | — |
| 103 | 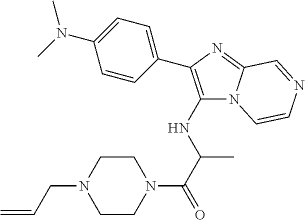 [SMILES: C=CCN1CCN(C(=O)C(C)Nc2c(-c3ccc(N(C)C)cc3)nc3cnccn23)CC1] | — |
| 104 | 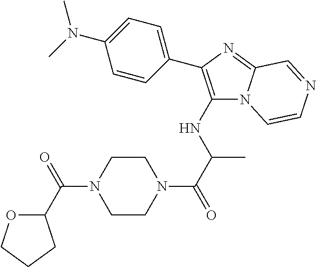 [SMILES: CC(Nc1c(-c2ccc(N(C)C)cc2)nc2cnccn12)C(=O)N1CCN(C(=O)C2CCCO2)CC1] | — |
| 105 | 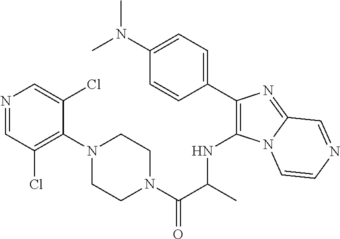 [SMILES: CC(Nc1c(-c2ccc(N(C)C)cc2)nc2cnccn12)C(=O)N1CCN(c2c(Cl)cncc2Cl)CC1] | — |
| 106 | 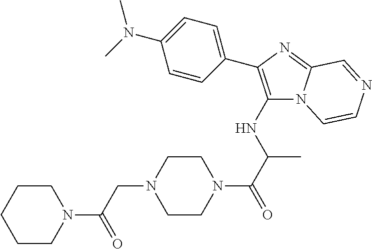 [SMILES: CC(Nc1c(-c2ccc(N(C)C)cc2)nc2cnccn12)C(=O)N1CCN(CC(=O)N2CCCCC2)CC1] | — |
| 107 | 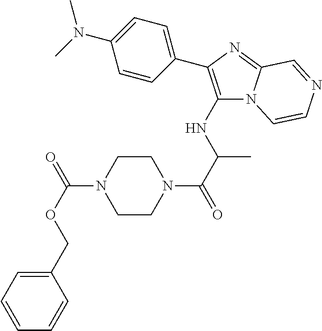 [SMILES: CC(Nc1c(-c2ccc(N(C)C)cc2)nc2cnccn12)C(=O)N1CCN(C(=O)OCc2ccccc2)CC1] | — |
| 108 | 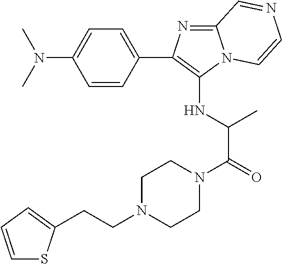 [SMILES: CC(Nc1c(-c2ccc(N(C)C)cc2)nc2cnccn12)C(=O)N1CCN(CCc2cccs2)CC1] | — |
| 109 | 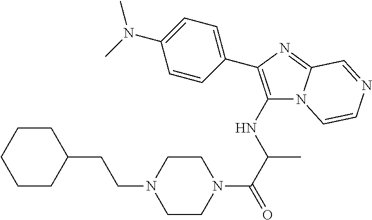 [SMILES: CC(Nc1c(-c2ccc(N(C)C)cc2)nc2cnccn12)C(=O)N1CCN(CCC2CCCCC2)CC1] | — |
| 110 | 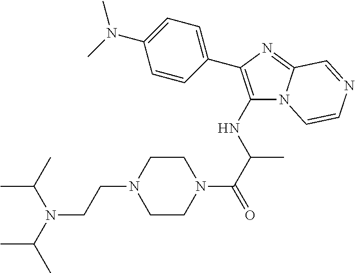 [SMILES: CC(Nc1c(-c2ccc(N(C)C)cc2)nc2cnccn12)C(=O)N1CCN(CCN(C(C)C)C(C)C)CC1] | — |
| 111 | 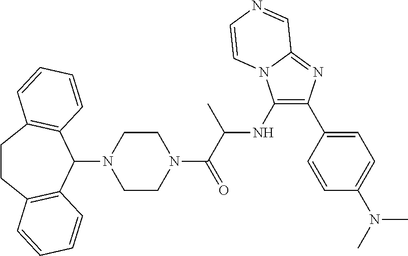 [SMILES: CC(Nc1c(-c2ccc(N(C)C)cc2)nc2cnccn12)C(=O)N1CCN(C2c3ccccc3CCc3ccccc32)CC1] | — |
| 112 | 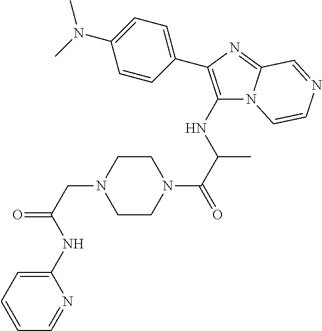 [SMILES: CC(Nc1c(-c2ccc(N(C)C)cc2)nc2cnccn12)C(=O)N1CCN(CC(=O)Nc2ccccn2)CC1] | — |
| 113 | 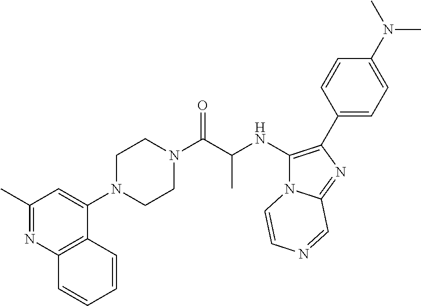 | — |
| 114 | 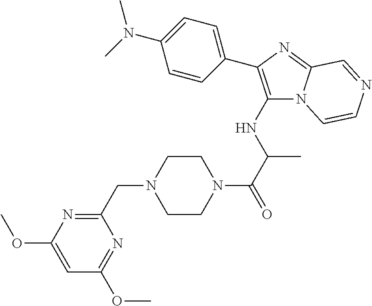 [SMILES: COc1cc(OC)nc(CN2CCN(C(=O)C(C)Nc3c(-c4ccc(N(C)C)cc4)nc4cnccn34)CC2)n1] | — |
| 115 | 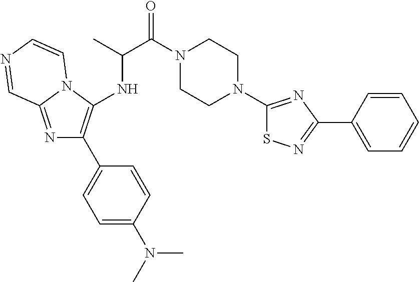 [SMILES: CC(Nc1c(-c2ccc(N(C)C)cc2)nc2cnccn12)C(=O)N1CCN(c2nc(-c3ccccc3)ns2)CC1] | — |
| 116 | 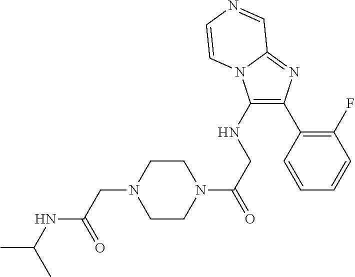 [SMILES: CC(C)NC(=O)CN1CCN(C(=O)CNc2c(-c3ccccc3F)nc3cnccn23)CC1] | — |
| 117 | 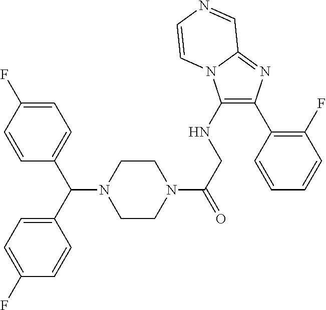 [SMILES: O=C(CNc1c(-c2ccccc2F)nc2cnccn12)N1CCN(C(c2ccc(F)cc2)c2ccc(F)cc2)CC1] | — |
| 118 | 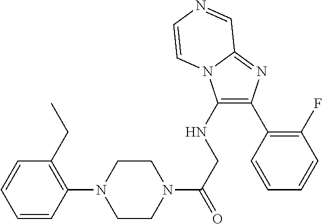 [SMILES: CCc1ccccc1N1CCN(C(=O)CNc2c(-c3ccccc3F)nc3cnccn23)CC1] | — |
| 119 | 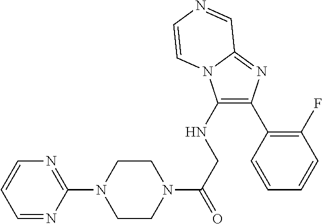 [SMILES: O=C(CNc1c(-c2ccccc2F)nc2cnccn12)N1CCN(c2ncccn2)CC1] | — |
| 120 | 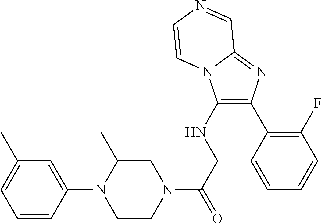 [SMILES: Cc1cccc(N2CCN(C(=O)CNc3c(-c4ccccc4F)nc4cnccn34)CC2C)c1] | — |
| 121 | 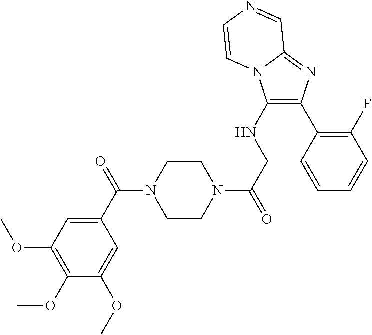 [SMILES: COc1cc(C(=O)N2CCN(C(=O)CNc3c(-c4ccccc4F)nc4cnccn34)CC2)cc(OC)c1OC] | — |
| 122 | 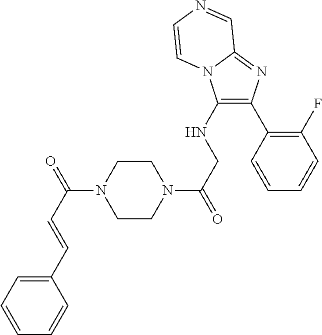 [SMILES: O=C(/C=C/c1ccccc1)N1CCN(C(=O)CNc2c(-c3ccccc3F)nc3cnccn23)CC1] | — |
| 123 | 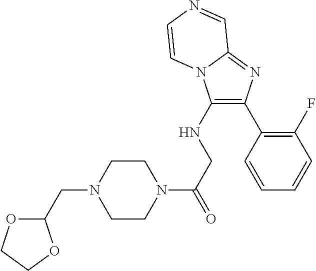 [SMILES: O=C(CNc1c(-c2ccccc2F)nc2cnccn12)N1CCN(CC2OCCO2)CC1] | — |
| 124 | 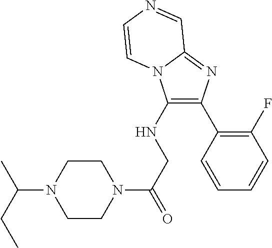 [SMILES: CCC(C)N1CCN(C(=O)CNc2c(-c3ccccc3F)nc3cnccn23)CC1] | — |
| 125 | 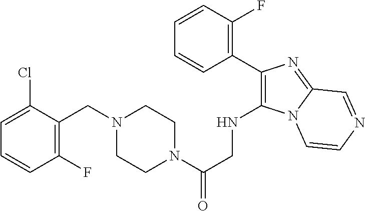 [SMILES: O=C(CNc1c(-c2ccccc2F)nc2cnccn12)N1CCN(Cc2c(F)cccc2Cl)CC1] | — |
| 126 | 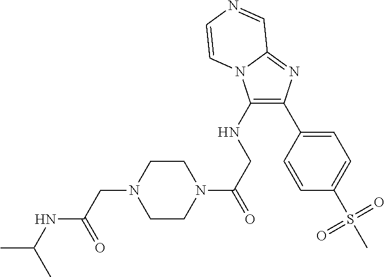 [SMILES: CC(C)NC(=O)CN1CCN(C(=O)CNc2c(-c3ccc(S(C)(=O)=O)cc3)nc3cnccn23)CC1] | — |
| 127 | 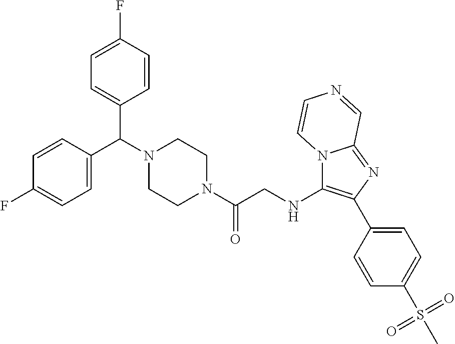 [SMILES: CS(=O)(=O)c1ccc(-c2nc3cnccn3c2NCC(=O)N2CCN(C(c3ccc(F)cc3)c3ccc(F)cc3)CC2)cc1] | — |
| 128 | 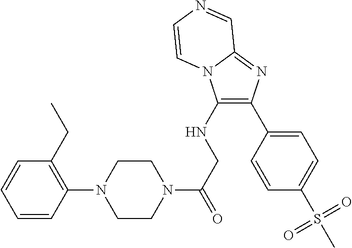 [SMILES: CCc1ccccc1N1CCN(C(=O)CNc2c(-c3ccc(S(C)(=O)=O)cc3)nc3cnccn23)CC1] | — |
| 129 | 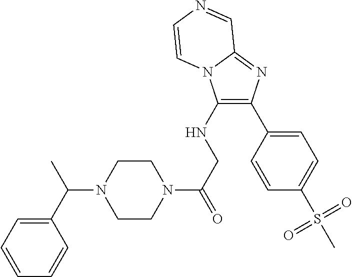 [SMILES: CC(c1ccccc1)N1CCN(C(=O)CNc2c(-c3ccc(S(C)(=O)=O)cc3)nc3cnccn23)CC1] | — |
| 130 | 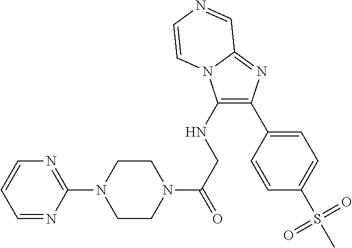 [SMILES: CS(=O)(=O)c1ccc(-c2nc3cnccn3c2NCC(=O)N2CCN(c3ncccn3)CC2)cc1] | — |
| 131 | 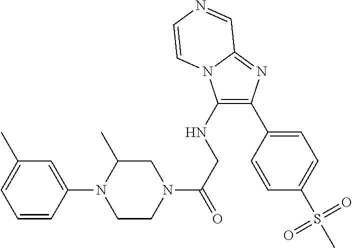 [SMILES: Cc1cccc(N2CCN(C(=O)CNc3c(-c4ccc(S(C)(=O)=O)cc4)nc4cnccn34)CC2C)c1] | — |
| 132 | 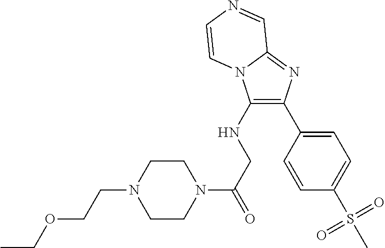 [SMILES: CCOCCN1CCN(C(=O)CNc2c(-c3ccc(S(C)(=O)=O)cc3)nc3cnccn23)CC1] | — |
| 133 | 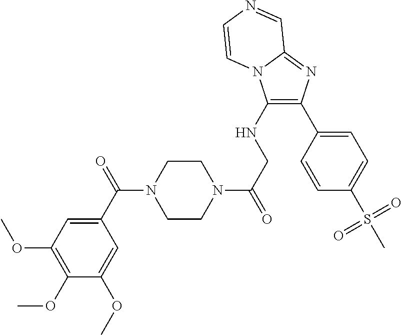 [SMILES: COc1cc(C(=O)N2CCN(C(=O)CNc3c(-c4ccc(S(C)(=O)=O)cc4)nc4cnccn34)CC2)cc(OC)c1OC] | — |
| 134 | 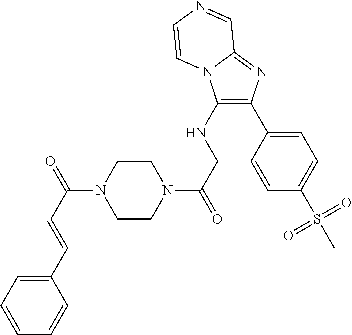 [SMILES: CS(=O)(=O)c1ccc(-c2nc3cnccn3c2NCC(=O)N2CCN(C(=O)/C=C/c3ccccc3)CC2)cc1] | — |
| 135 | 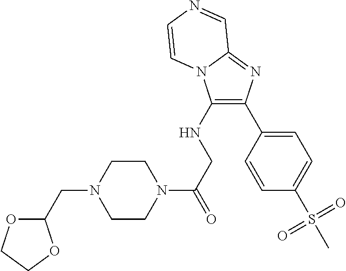 [SMILES: CS(=O)(=O)c1ccc(-c2nc3cnccn3c2NCC(=O)N2CCN(CC3OCCO3)CC2)cc1] | — |
| 136 | 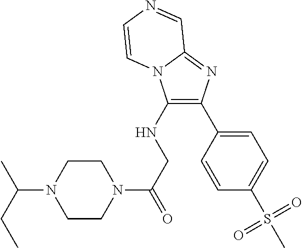 [SMILES: CCC(C)N1CCN(C(=O)CNc2c(-c3ccc(S(C)(=O)=O)cc3)nc3cnccn23)CC1] | — |
| 137 | 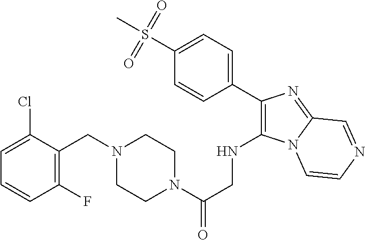 [SMILES: CS(=O)(=O)c1ccc(-c2nc3cnccn3c2NCC(=O)N2CCN(Cc3c(F)cccc3Cl)CC2)cc1] | — |
| 138 | 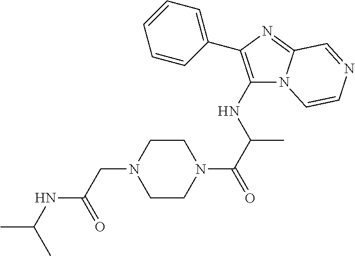 [SMILES: CC(C)NC(=O)CN1CCN(C(=O)C(C)Nc2c(-c3ccccc3)nc3cnccn23)CC1] | — |
| 139 | 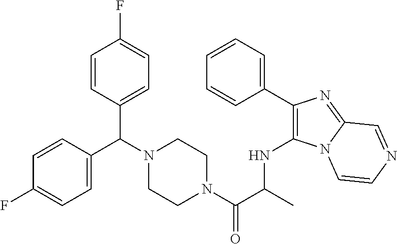 [SMILES: CC(Nc1c(-c2ccccc2)nc2cnccn12)C(=O)N1CCN(C(c2ccc(F)cc2)c2ccc(F)cc2)CC1] | — |
| 140 | 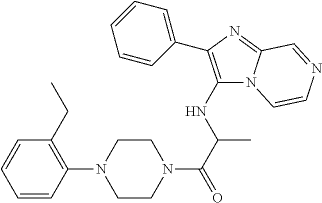 [SMILES: CCc1ccccc1N1CCN(C(=O)C(C)Nc2c(-c3ccccc3)nc3cnccn23)CC1] | — |
| 141 | 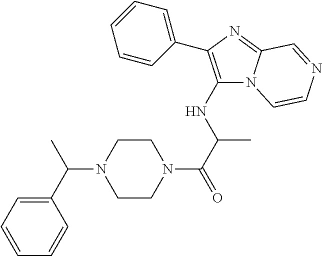 [SMILES: CC(Nc1c(-c2ccccc2)nc2cnccn12)C(=O)N1CCN(C(C)c2ccccc2)CC1] | — |
| 142 | 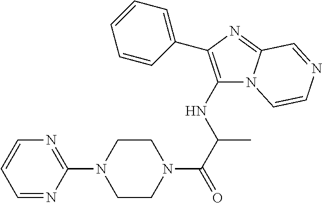 [SMILES: CC(Nc1c(-c2ccccc2)nc2cnccn12)C(=O)N1CCN(c2ncccn2)CC1] | — |
| 143 | 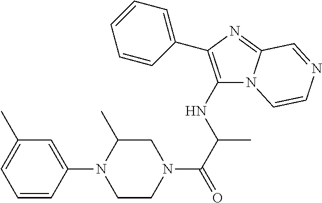 [SMILES: Cc1cccc(N2CCN(C(=O)C(C)Nc3c(-c4ccccc4)nc4cnccn34)CC2C)c1] | — |
| 144 | 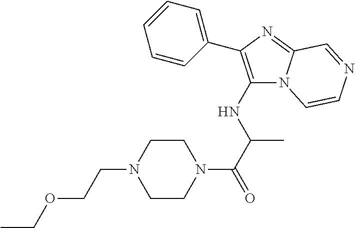 [SMILES: CCOCCN1CCN(C(=O)C(C)Nc2c(-c3ccccc3)nc3cnccn23)CC1] | — |
| 145 | 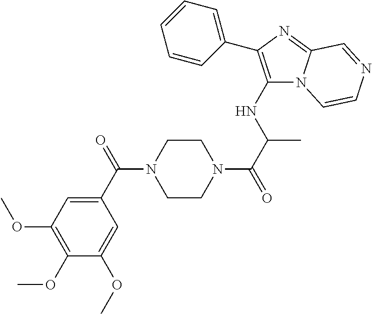 [SMILES: COc1cc(C(=O)N2CCN(C(=O)C(C)Nc3c(-c4ccccc4)nc4cnccn34)CC2)cc(OC)c1OC] | — |
| 146 | 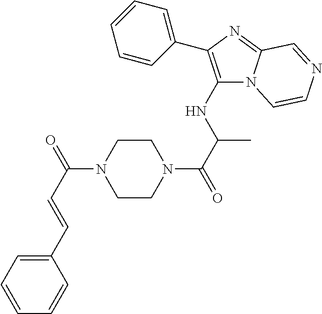 [SMILES: CC(Nc1c(-c2ccccc2)nc2cnccn12)C(=O)N1CCN(C(=O)/C=C/c2ccccc2)CC1] | — |
| 147 | 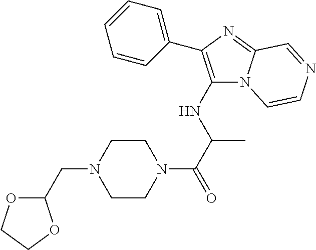 [SMILES: CC(Nc1c(-c2ccccc2)nc2cnccn12)C(=O)N1CCN(CC2OCCO2)CC1] | — |
| 148 | 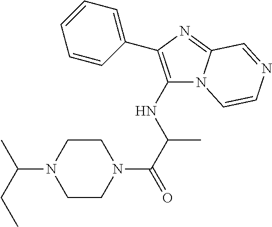 [SMILES: CCC(C)N1CCN(C(=O)C(C)Nc2c(-c3ccccc3)nc3cnccn23)CC1] | — |
| 149 | 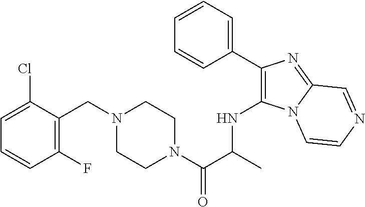 [SMILES: CC(Nc1c(-c2ccccc2)nc2cnccn12)C(=O)N1CCN(Cc2c(F)cccc2Cl)CC1] | — |
| 150 | 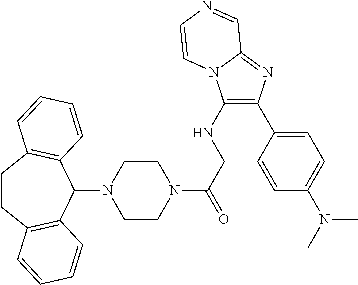 [SMILES: CN(C)c1ccc(-c2nc3cnccn3c2NCC(=O)N2CCN(C3c4ccccc4CCc4ccccc43)CC2)cc1] | — |
| 151 | 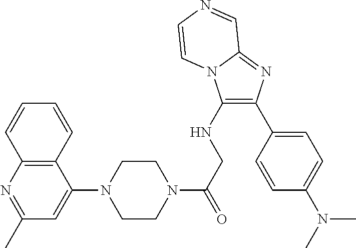 [SMILES: Cc1cc(N2CCN(C(=O)CNc3c(-c4ccc(N(C)C)cc4)nc4cnccn34)CC2)c2ccccc2n1] | — |
| 152 | 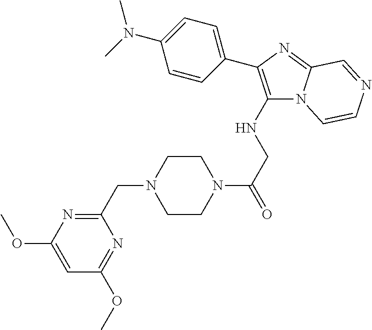 [SMILES: COc1cc(OC)nc(CN2CCN(C(=O)CNc3c(-c4ccc(N(C)C)cc4)nc4cnccn34)CC2)n1] | — |
| 153 | 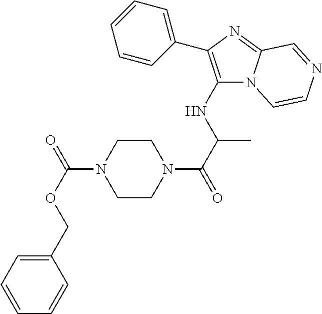 [SMILES: CC(Nc1c(-c2ccccc2)nc2cnccn12)C(=O)N1CCN(C(=O)OCc2ccccc2)CC1] | — |
| 154 | 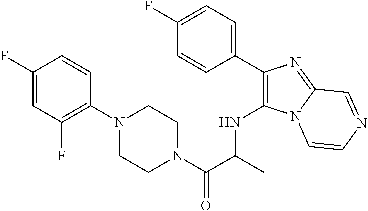 [SMILES: CC(Nc1c(-c2ccc(F)cc2)nc2cnccn12)C(=O)N1CCN(c2ccc(F)cc2F)CC1] | — |
| 155 | 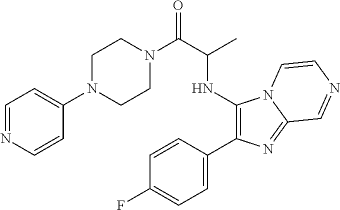 [SMILES: CC(Nc1c(-c2ccc(F)cc2)nc2cnccn12)C(=O)N1CCN(c2ccncc2)CC1] | — |
| 156 | 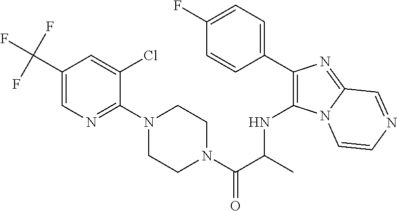 [SMILES: CC(Nc1c(-c2ccc(F)cc2)nc2cnccn12)C(=O)N1CCN(c2ncc(C(F)(F)F)cc2Cl)CC1] | — |
| 157 | 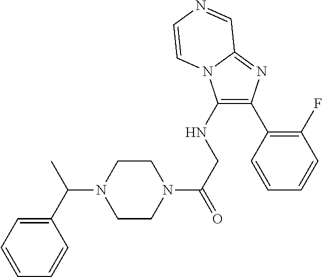 [SMILES: CC(c1ccccc1)N1CCN(C(=O)CNc2c(-c3ccccc3F)nc3cnccn23)CC1] | — |
| 158 | 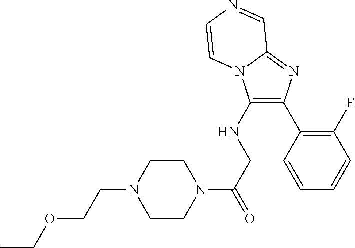 [SMILES: CCOCCN1CCN(C(=O)CNc2c(-c3ccccc3F)nc3cnccn23)CC1] | — |
| 159 | 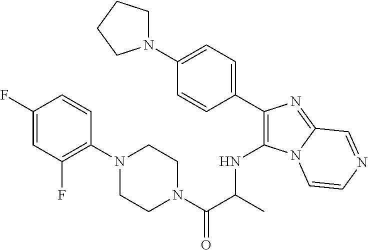 [SMILES: CC(Nc1c(-c2ccc(N3CCCC3)cc2)nc2cnccn12)C(=O)N1CCN(c2ccc(F)cc2F)CC1] | — |
| 160 | 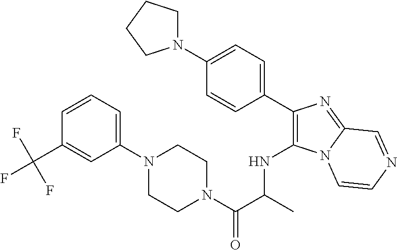 [SMILES: CC(Nc1c(-c2ccc(N3CCCC3)cc2)nc2cnccn12)C(=O)N1CCN(c2cccc(C(F)(F)F)c2)CC1] | — |
| 161 | 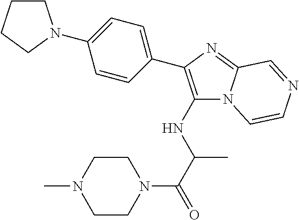 [SMILES: CC(Nc1c(-c2ccc(N3CCCC3)cc2)nc2cnccn12)C(=O)N1CCN(C)CC1] | — |
| 162 | 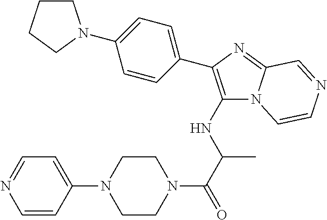 [SMILES: CC(Nc1c(-c2ccc(N3CCCC3)cc2)nc2cnccn12)C(=O)N1CCN(c2ccncc2)CC1] | — |
| 163 | 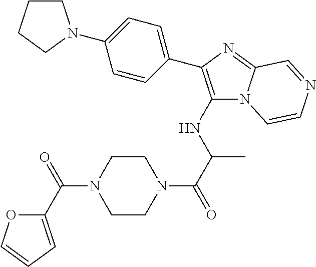 [SMILES: CC(Nc1c(-c2ccc(N3CCCC3)cc2)nc2cnccn12)C(=O)N1CCN(C(=O)c2ccco2)CC1] | — |
| 164 | 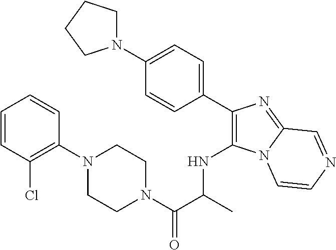 [SMILES: CC(Nc1c(-c2ccc(N3CCCC3)cc2)nc2cnccn12)C(=O)N1CCN(c2ccccc2Cl)CC1] | — |
| 165 | 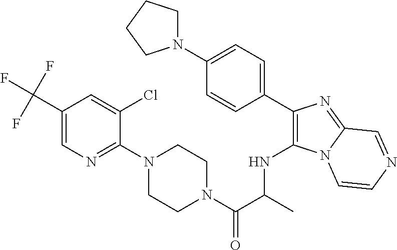 [SMILES: CC(Nc1c(-c2ccc(N3CCCC3)cc2)nc2cnccn12)C(=O)N1CCN(c2ncc(C(F)(F)F)cc2Cl)CC1] | — |
| 166 | 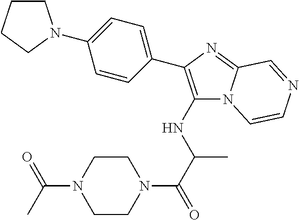 [SMILES: CC(=O)N1CCN(C(=O)C(C)Nc2c(-c3ccc(N4CCCC4)cc3)nc3cnccn23)CC1] | — |
| 167 | 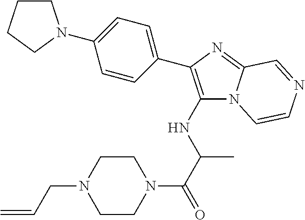 [SMILES: C=CCN1CCN(C(=O)C(C)Nc2c(-c3ccc(N4CCCC4)cc3)nc3cnccn23)CC1] | — |
| 168 | 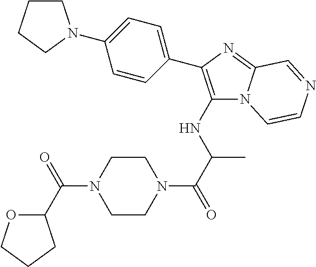 [SMILES: CC(Nc1c(-c2ccc(N3CCCC3)cc2)nc2cnccn12)C(=O)N1CCN(C(=O)C2CCCO2)CC1] | — |
| 169 | 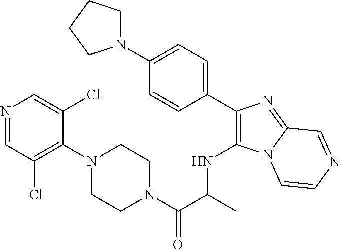 [SMILES: CC(Nc1c(-c2ccc(N3CCCC3)cc2)nc2cnccn12)C(=O)N1CCN(c2c(Cl)cncc2Cl)CC1] | — |
| 170 | 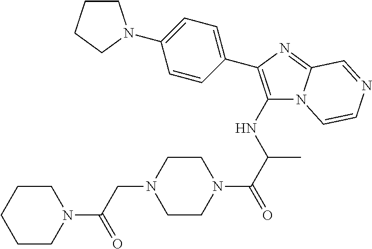 [SMILES: CC(Nc1c(-c2ccc(N3CCCC3)cc2)nc2cnccn12)C(=O)N1CCN(CC(=O)N2CCCCC2)CC1] | — |
| 171 | 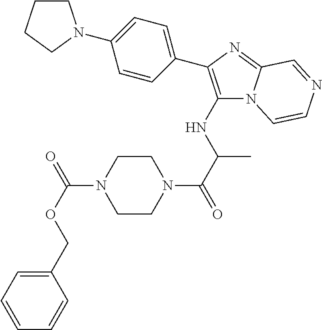 [SMILES: CC(Nc1c(-c2ccc(N3CCCC3)cc2)nc2cnccn12)C(=O)N1CCN(C(=O)OCc2ccccc2)CC1] | — |
| 172 | 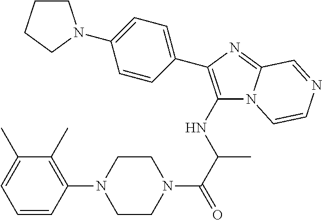 [SMILES: Cc1cccc(N2CCN(C(=O)C(C)Nc3c(-c4ccc(N5CCCC5)cc4)nc4cnccn34)CC2)c1C] | — |
| 173 | 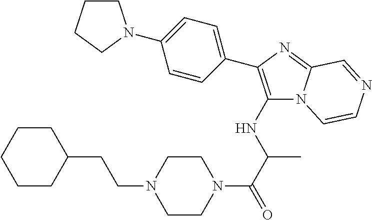 [SMILES: CC(Nc1c(-c2ccc(N3CCCC3)cc2)nc2cnccn12)C(=O)N1CCN(CCC2CCCCC2)CC1] | — |
| 174 | 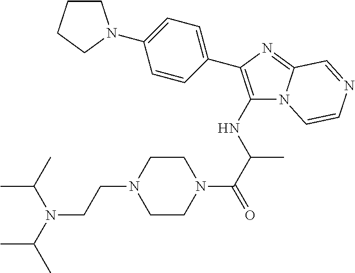 [SMILES: CC(Nc1c(-c2ccc(N3CCCC3)cc2)nc2cnccn12)C(=O)N1CCN(CCN(C(C)C)C(C)C)CC1] | — |
| 175 | 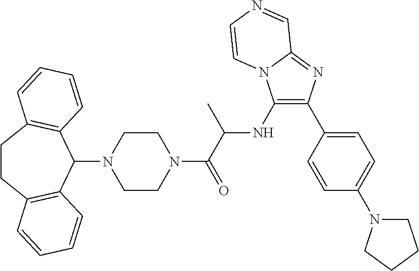 [SMILES: CC(Nc1c(-c2ccc(N3CCCC3)cc2)nc2cnccn12)C(=O)N1CCN(C2c3ccccc3CCc3ccccc32)CC1] | — |
| 176 | 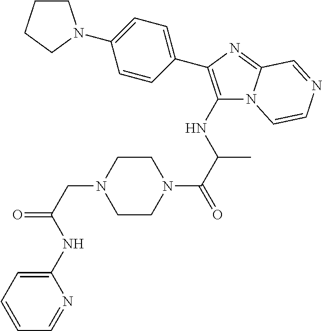 [SMILES: CC(Nc1c(-c2ccc(N3CCCC3)cc2)nc2cnccn12)C(=O)N1CCN(CC(=O)Nc2ccccn2)CC1] | — |
| 177 | 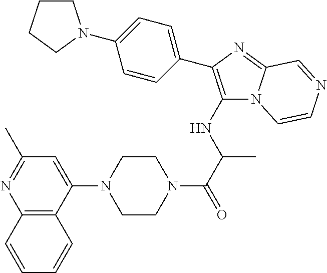 [SMILES: Cc1cc(N2CCN(C(=O)C(C)Nc3c(-c4ccc(N5CCCC5)cc4)nc4cnccn34)CC2)c2ccccc2n1] | — |
| 178 | 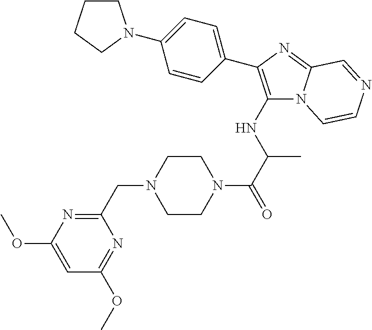 [SMILES: COc1cc(OC)nc(CN2CCN(C(=O)C(C)Nc3c(-c4ccc(N5CCCC5)cc4)nc4cnccn34)CC2)n1] | — |
| 179 | 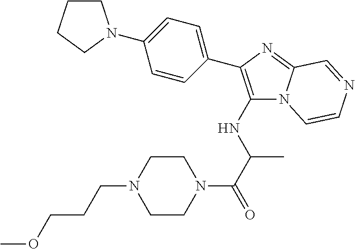 [SMILES: COCCCN1CCN(C(=O)C(C)Nc2c(-c3ccc(N4CCCC4)cc3)nc3cnccn23)CC1] | — |
| 180 | 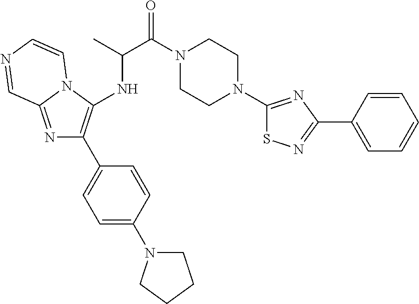 [SMILES: CC(Nc1c(-c2ccc(N3CCCC3)cc2)nc2cnccn12)C(=O)N1CCN(c2nc(-c3ccccc3)ns2)CC1] | — |
| 181 | 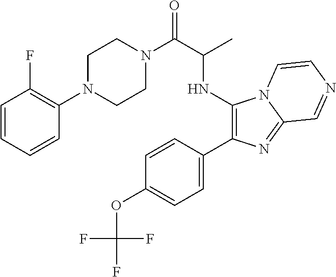 [SMILES: CC(Nc1c(-c2ccc(OC(F)(F)F)cc2)nc2cnccn12)C(=O)N1CCN(c2ccccc2F)CC1] | — |
| 182 | 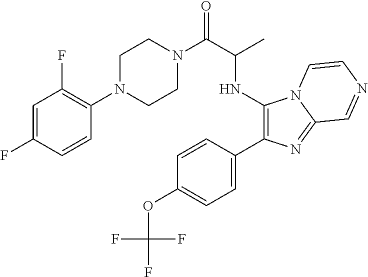 [SMILES: CC(Nc1c(-c2ccc(OC(F)(F)F)cc2)nc2cnccn12)C(=O)N1CCN(c2ccc(F)cc2F)CC1] | — |
| 183 | 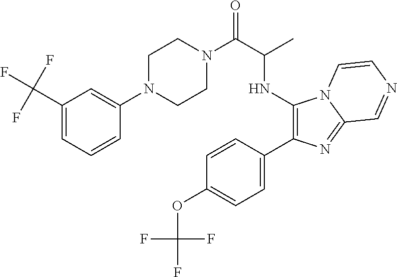 [SMILES: CC(Nc1c(-c2ccc(OC(F)(F)F)cc2)nc2cnccn12)C(=O)N1CCN(c2cccc(C(F)(F)F)c2)CC1] | — |
| 184 | 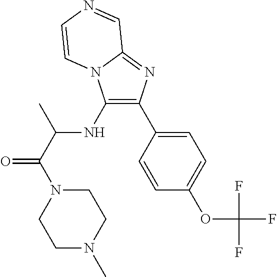 [SMILES: CC(Nc1c(-c2ccc(OC(F)(F)F)cc2)nc2cnccn12)C(=O)N1CCN(C)CC1] | — |
| 185 | 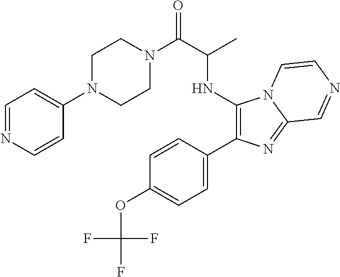 [SMILES: CC(Nc1c(-c2ccc(OC(F)(F)F)cc2)nc2cnccn12)C(=O)N1CCN(c2ccncc2)CC1] | — |
| 186 | 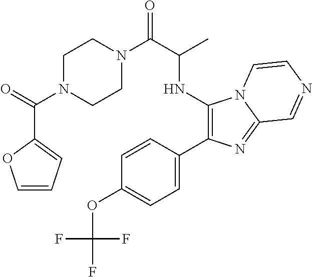 [SMILES: CC(Nc1c(-c2ccc(OC(F)(F)F)cc2)nc2cnccn12)C(=O)N1CCN(C(=O)c2ccco2)CC1] | — |
| 187 | 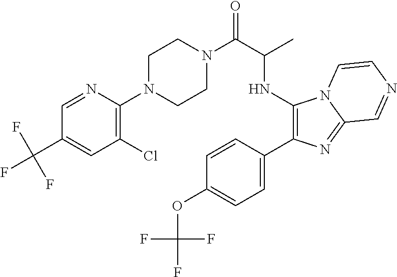 [SMILES: CC(Nc1c(-c2ccc(OC(F)(F)F)cc2)nc2cnccn12)C(=O)N1CCN(c2ncc(C(F)(F)F)cc2Cl)CC1] | — |
| 188 | 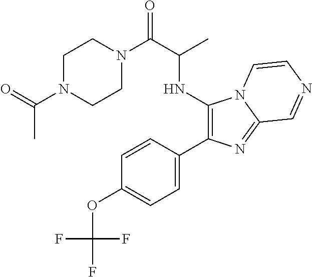 [SMILES: CC(=O)N1CCN(C(=O)C(C)Nc2c(-c3ccc(OC(F)(F)F)cc3)nc3cnccn23)CC1] | — |
| 189 | 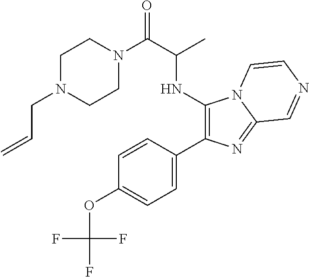 [SMILES: C=CCN1CCN(C(=O)C(C)Nc2c(-c3ccc(OC(F)(F)F)cc3)nc3cnccn23)CC1] | — |
| 190 | 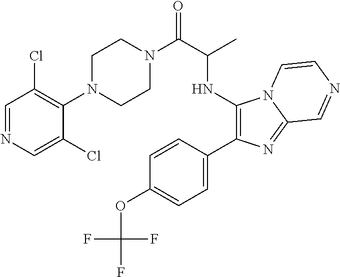 [SMILES: CC(Nc1c(-c2ccc(OC(F)(F)F)cc2)nc2cnccn12)C(=O)N1CCN(c2c(Cl)cncc2Cl)CC1] | — |
| 191 | 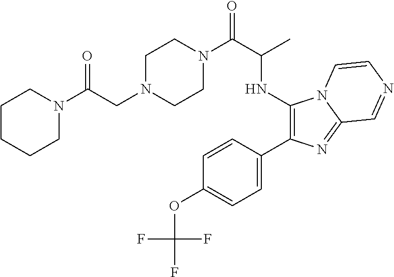 [SMILES: CC(Nc1c(-c2ccc(OC(F)(F)F)cc2)nc2cnccn12)C(=O)N1CCN(CC(=O)N2CCCCC2)CC1] | — |
| 192 | 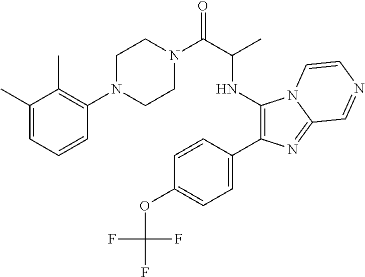 [SMILES: Cc1cccc(N2CCN(C(=O)C(C)Nc3c(-c4ccc(OC(F)(F)F)cc4)nc4cnccn34)CC2)c1C] | — |
| 193 | 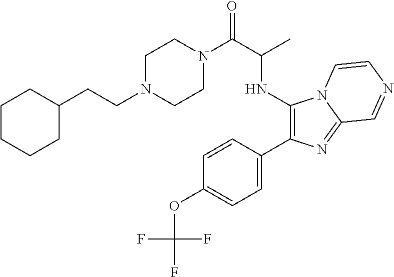 [SMILES: CC(Nc1c(-c2ccc(OC(F)(F)F)cc2)nc2cnccn12)C(=O)N1CCN(CCC2CCCCC2)CC1] | — |
| 194 | 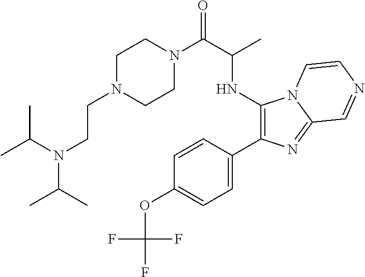 [SMILES: CC(Nc1c(-c2ccc(OC(F)(F)F)cc2)nc2cnccn12)C(=O)N1CCN(CCN(C(C)C)C(C)C)CC1] | — |
| 195 | 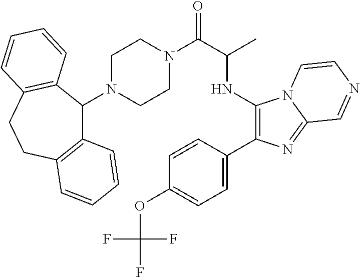 [SMILES: CC(Nc1c(-c2ccc(OC(F)(F)F)cc2)nc2cnccn12)C(=O)N1CCN(C2c3ccccc3CCc3ccccc32)CC1] | — |
| 196 | 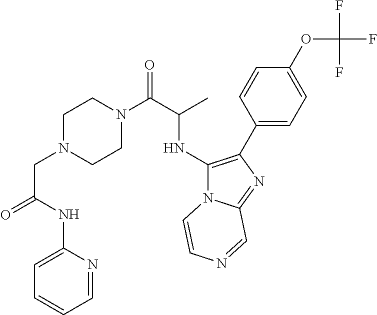 [SMILES: CC(Nc1c(-c2ccc(OC(F)(F)F)cc2)nc2cnccn12)C(=O)N1CCN(CC(=O)Nc2ccccn2)CC1] | — |
| 197 | 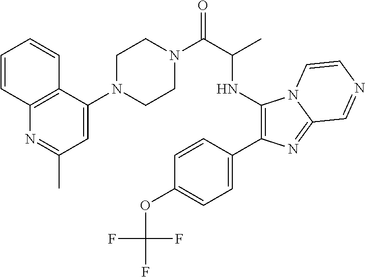 [SMILES: Cc1cc(N2CCN(C(=O)C(C)Nc3c(-c4ccc(OC(F)(F)F)cc4)nc4cnccn34)CC2)c2ccccc2n1] | — |
| 198 | 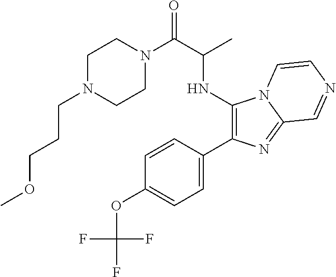 [SMILES: COCCCN1CCN(C(=O)C(C)Nc2c(-c3ccc(OC(F)(F)F)cc3)nc3cnccn23)CC1] | — |
| 199 | 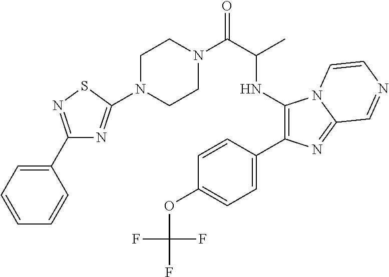 [SMILES: CC(Nc1c(-c2ccc(OC(F)(F)F)cc2)nc2cnccn12)C(=O)N1CCN(c2nc(-c3ccccc3)ns2)CC1] | — |
| 200 | 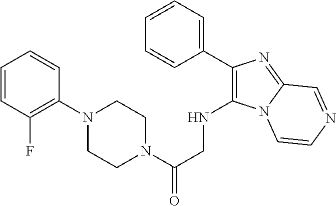 [SMILES: O=C(CNc1c(-c2ccccc2)nc2cnccn12)N1CCN(c2ccccc2F)CC1] | — |
| 201 | 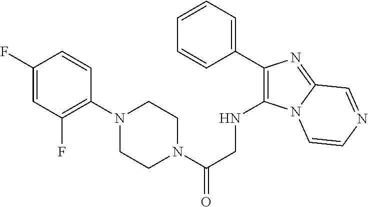 [SMILES: O=C(CNc1c(-c2ccccc2)nc2cnccn12)N1CCN(c2ccc(F)cc2F)CC1] | — |
| 202 | 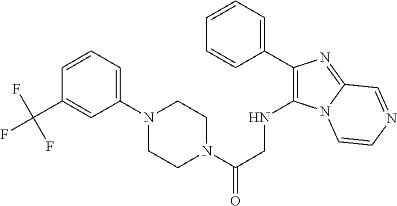 [SMILES: O=C(CNc1c(-c2ccccc2)nc2cnccn12)N1CCN(c2cccc(C(F)(F)F)c2)CC1] | — |
| 203 | 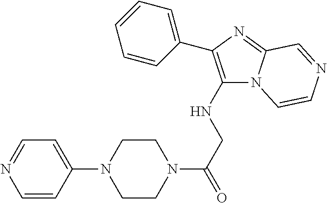 [SMILES: O=C(CNc1c(-c2ccccc2)nc2cnccn12)N1CCN(c2ccncc2)CC1] | — |
| 204 | 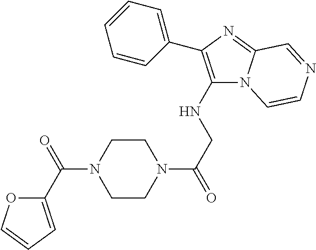 [SMILES: O=C(CNc1c(-c2ccccc2)nc2cnccn12)N1CCN(C(=O)c2ccco2)CC1] | — |
| 205 | 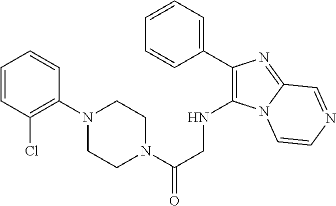 [SMILES: O=C(CNc1c(-c2ccccc2)nc2cnccn12)N1CCN(c2ccccc2Cl)CC1] | — |
| 206 | 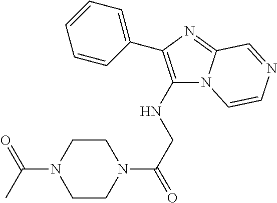 [SMILES: CC(=O)N1CCN(C(=O)CNc2c(-c3ccccc3)nc3cnccn23)CC1] | — |
| 207 | 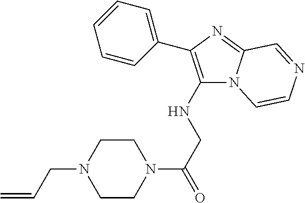 [SMILES: C=CCN1CCN(C(=O)CNc2c(-c3ccccc3)nc3cnccn23)CC1] | — |
| 208 | 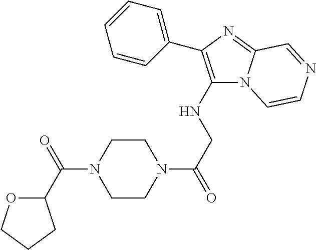 [SMILES: O=C(CNc1c(-c2ccccc2)nc2cnccn12)N1CCN(C(=O)C2CCCO2)CC1] | — |
| 209 | 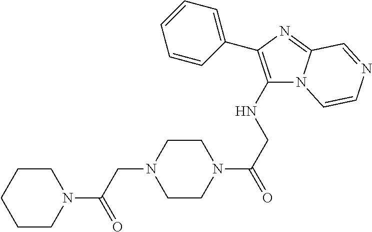 [SMILES: O=C(CNc1c(-c2ccccc2)nc2cnccn12)N1CCN(CC(=O)N2CCCCC2)CC1] | — |
| 210 | 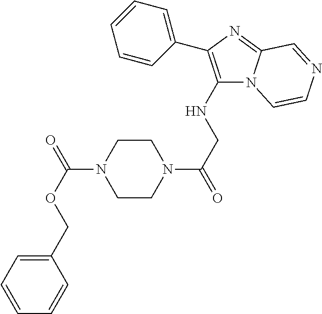 [SMILES: O=C(CNc1c(-c2ccccc2)nc2cnccn12)N1CCN(C(=O)OCc2ccccc2)CC1] | — |
| 211 | 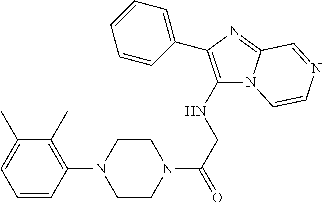 [SMILES: Cc1cccc(N2CCN(C(=O)CNc3c(-c4ccccc4)nc4cnccn34)CC2)c1C] | — |
| 212 | 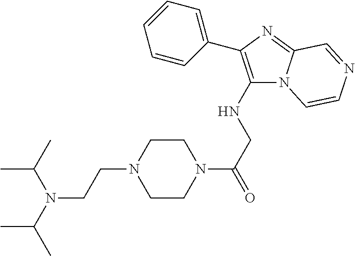 [SMILES: CC(C)N(CCN1CCN(C(=O)CNc2c(-c3ccccc3)nc3cnccn23)CC1)C(C)C] | — |
| 213 | 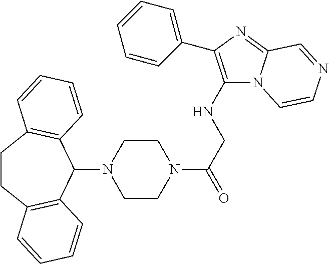 [SMILES: O=C(CNc1c(-c2ccccc2)nc2cnccn12)N1CCN(C2c3ccccc3CCc3ccccc32)CC1] | — |
| 214 | 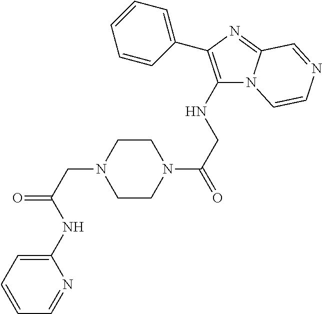 [SMILES: O=C(CN1CCN(C(=O)CNc2c(-c3ccccc3)nc3cnccn23)CC1)Nc1ccccn1] | — |
| 215 | 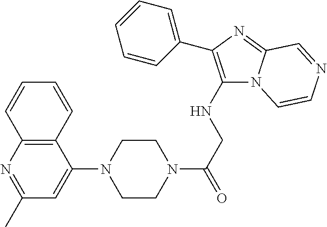 [SMILES: Cc1cc(N2CCN(C(=O)CNc3c(-c4ccccc4)nc4cnccn34)CC2)c2ccccc2n1] | — |
| 216 | 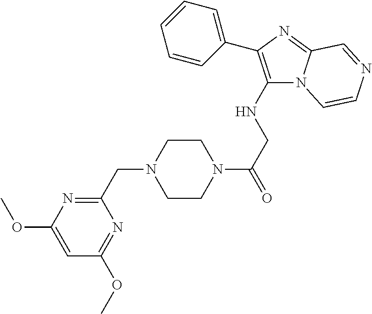 [SMILES: COc1cc(OC)nc(CN2CCN(C(=O)CNc3c(-c4ccccc4)nc4cnccn34)CC2)n1] | — |
| 217 | 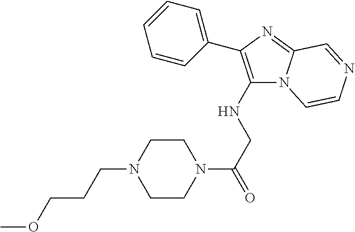 [SMILES: COCCCN1CCN(C(=O)CNc2c(-c3ccccc3)nc3cnccn23)CC1] | — |
| 218 | 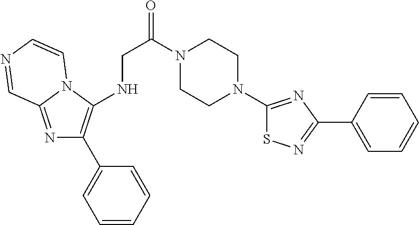 [SMILES: O=C(CNc1c(-c2ccccc2)nc2cnccn12)N1CCN(c2nc(-c3ccccc3)ns2)CC1] | — |
| 219 | 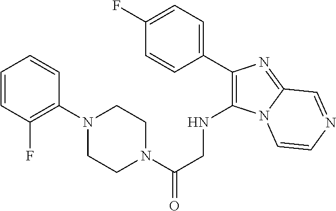 [SMILES: O=C(CNc1c(-c2ccc(F)cc2)nc2cnccn12)N1CCN(c2ccccc2F)CC1] | — |
| 220 | 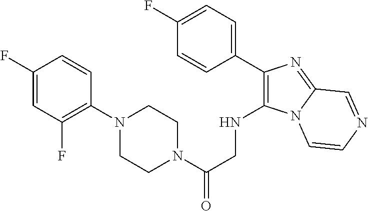 [SMILES: O=C(CNc1c(-c2ccc(F)cc2)nc2cnccn12)N1CCN(c2ccc(F)cc2F)CC1] | — |
| 221 | 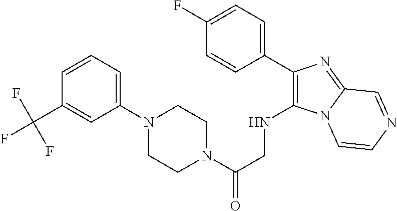 [SMILES: O=C(CNc1c(-c2ccc(F)cc2)nc2cnccn12)N1CCN(c2cccc(C(F)(F)F)c2)CC1] | — |
| 222 | 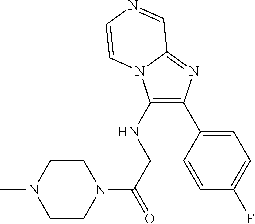 [SMILES: CN1CCN(C(=O)CNc2c(-c3ccc(F)cc3)nc3cnccn23)CC1] | — |
| 223 | 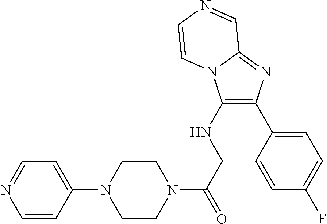 [SMILES: O=C(CNc1c(-c2ccc(F)cc2)nc2cnccn12)N1CCN(c2ccncc2)CC1] | — |
| 224 | 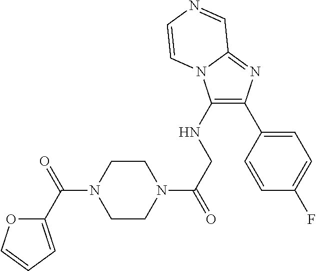 [SMILES: O=C(CNc1c(-c2ccc(F)cc2)nc2cnccn12)N1CCN(C(=O)c2ccco2)CC1] | — |
| 225 | 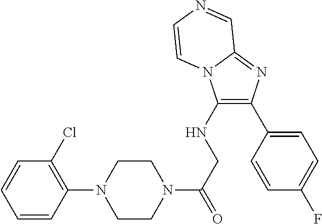 [SMILES: O=C(CNc1c(-c2ccc(F)cc2)nc2cnccn12)N1CCN(c2ccccc2Cl)CC1] | — |
| 226 | 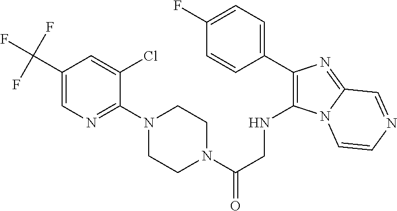 [SMILES: O=C(CNc1c(-c2ccc(F)cc2)nc2cnccn12)N1CCN(c2ncc(C(F)(F)F)cc2Cl)CC1] | — |
| 227 | 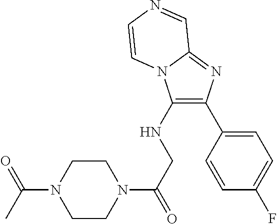 [SMILES: CC(=O)N1CCN(C(=O)CNc2c(-c3ccc(F)cc3)nc3cnccn23)CC1] | — |
| 228 | 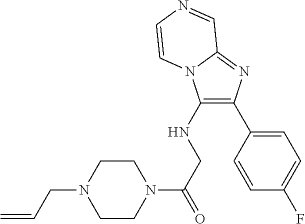 [SMILES: C=CCN1CCN(C(=O)CNc2c(-c3ccc(F)cc3)nc3cnccn23)CC1] | — |
| 229 | 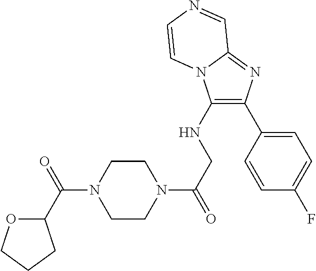 [SMILES: O=C(CNc1c(-c2ccc(F)cc2)nc2cnccn12)N1CCN(C(=O)C2CCCO2)CC1] | — |
| 230 | 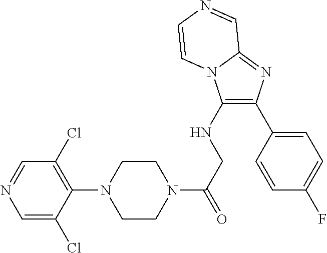 [SMILES: O=C(CNc1c(-c2ccc(F)cc2)nc2cnccn12)N1CCN(c2c(Cl)cncc2Cl)CC1] | — |
| 231 | 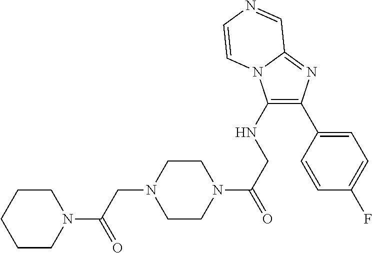 [SMILES: O=C(CNc1c(-c2ccc(F)cc2)nc2cnccn12)N1CCN(CC(=O)N2CCCCC2)CC1] | — |
| 232 | 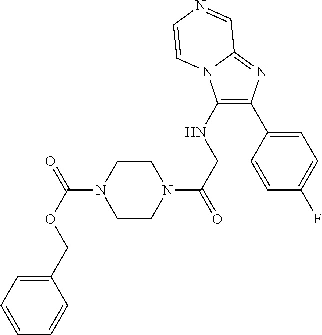 [SMILES: O=C(CNc1c(-c2ccc(F)cc2)nc2cnccn12)N1CCN(C(=O)OCc2ccccc2)CC1] | — |
| 233 | 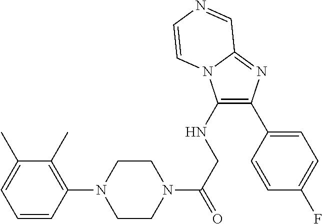 [SMILES: Cc1cccc(N2CCN(C(=O)CNc3c(-c4ccc(F)cc4)nc4cnccn34)CC2)c1C] | — |
| 234 | 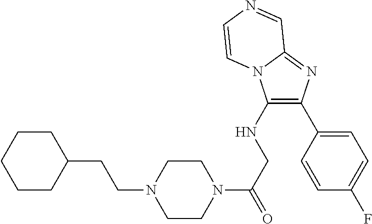 [SMILES: O=C(CNc1c(-c2ccc(F)cc2)nc2cnccn12)N1CCN(CCC2CCCCC2)CC1] | — |
| 235 | 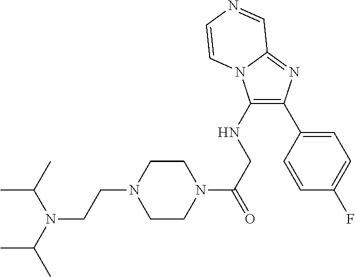 [SMILES: CC(C)N(CCN1CCN(C(=O)CNc2c(-c3ccc(F)cc3)nc3cnccn23)CC1)C(C)C] | — |
| 236 | 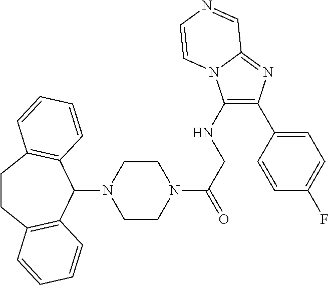 [SMILES: O=C(CNc1c(-c2ccc(F)cc2)nc2cnccn12)N1CCN(C2c3ccccc3CCc3ccccc32)CC1] | — |
| 237 | 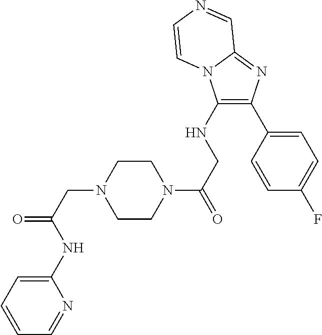 [SMILES: O=C(CN1CCN(C(=O)CNc2c(-c3ccc(F)cc3)nc3cnccn23)CC1)Nc1ccccn1] | — |
| 238 | 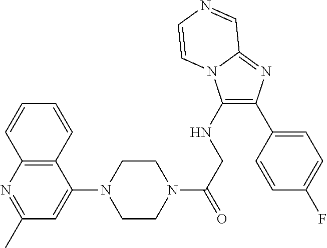 [SMILES: Cc1cc(N2CCN(C(=O)CNc3c(-c4ccc(F)cc4)nc4cnccn34)CC2)c2ccccc2n1] | — |
| 239 | 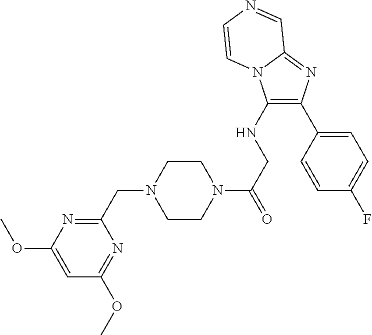 [SMILES: COc1cc(OC)nc(CN2CCN(C(=O)CNc3c(-c4ccc(F)cc4)nc4cnccn34)CC2)n1] | — |
| 240 | 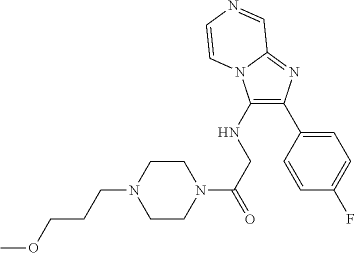 [SMILES: COCCCN1CCN(C(=O)CNc2c(-c3ccc(F)cc3)nc3cnccn23)CC1] | — |
| 241 | 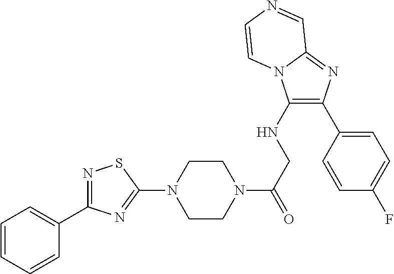 [SMILES: O=C(CNc1c(-c2ccc(F)cc2)nc2cnccn12)N1CCN(c2nc(-c3ccccc3)ns2)CC1] | — |
| 242 | 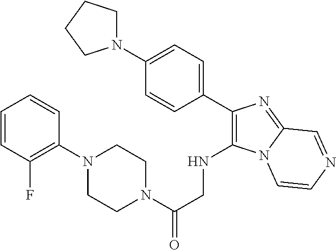 [SMILES: O=C(CNc1c(-c2ccc(N3CCCC3)cc2)nc2cnccn12)N1CCN(c2ccccc2F)CC1] | — |
| 243 | 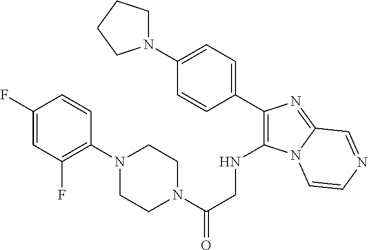 [SMILES: O=C(CNc1c(-c2ccc(N3CCCC3)cc2)nc2cnccn12)N1CCN(c2ccc(F)cc2F)CC1] | — |
| 244 | 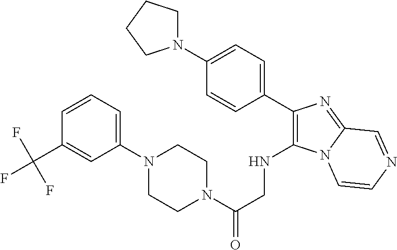 [SMILES: O=C(CNc1c(-c2ccc(N3CCCC3)cc2)nc2cnccn12)N1CCN(c2cccc(C(F)(F)F)c2)CC1] | — |
| 245 | 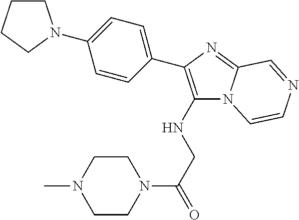 [SMILES: CN1CCN(C(=O)CNc2c(-c3ccc(N4CCCC4)cc3)nc3cnccn23)CC1] | — |
| 246 | 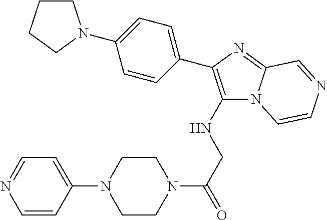 [SMILES: O=C(CNc1c(-c2ccc(N3CCCC3)cc2)nc2cnccn12)N1CCN(c2ccncc2)CC1] | — |
| 247 | 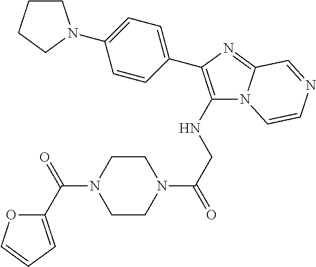 [SMILES: O=C(CNc1c(-c2ccc(N3CCCC3)cc2)nc2cnccn12)N1CCN(C(=O)c2ccco2)CC1] | — |
| 248 | 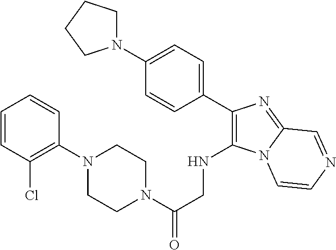 [SMILES: O=C(CNc1c(-c2ccc(N3CCCC3)cc2)nc2cnccn12)N1CCN(c2ccccc2Cl)CC1] | 516.3 |
| 249 | 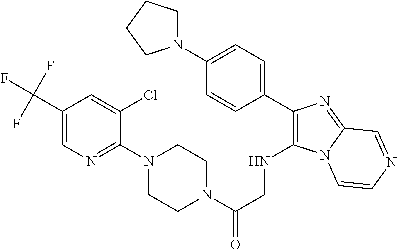 [SMILES: O=C(CNc1c(-c2ccc(N3CCCC3)cc2)nc2cnccn12)N1CCN(c2ncc(C(F)(F)F)cc2Cl)CC1] | — |
| 250 | 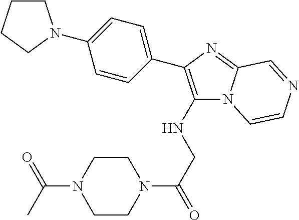 [SMILES: CC(=O)N1CCN(C(=O)CNc2c(-c3ccc(N4CCCC4)cc3)nc3cnccn23)CC1] | — |
| 251 | 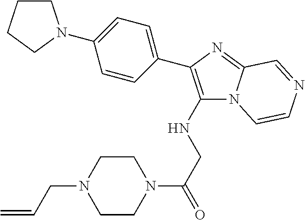 [SMILES: C=CCN1CCN(C(=O)CNc2c(-c3ccc(N4CCCC4)cc3)nc3cnccn23)CC1] | — |
| 252 | 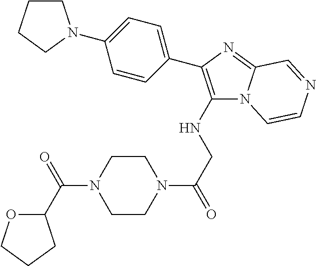 [SMILES: O=C(CNc1c(-c2ccc(N3CCCC3)cc2)nc2cnccn12)N1CCN(C(=O)C2CCCO2)CC1] | — |
| 253 | 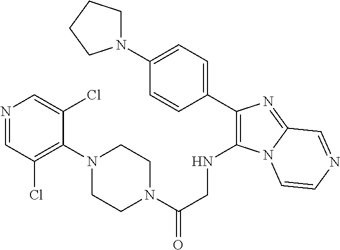 [SMILES: O=C(CNc1c(-c2ccc(N3CCCC3)cc2)nc2cnccn12)N1CCN(c2c(Cl)cncc2Cl)CC1] | 551.3 |
| 254 | 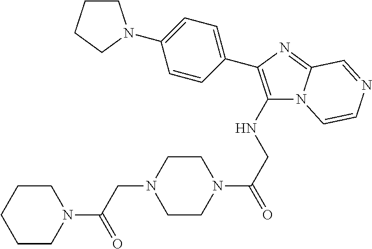 [SMILES: O=C(CNc1c(-c2ccc(N3CCCC3)cc2)nc2cnccn12)N1CCN(CC(=O)N2CCCCC2)CC1] | — |
| 255 | 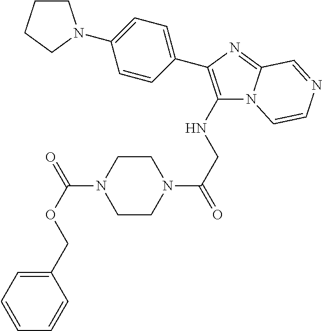 [SMILES: O=C(CNc1c(-c2ccc(N3CCCC3)cc2)nc2cnccn12)N1CCN(C(=O)OCc2ccccc2)CC1] | — |
| 256 | 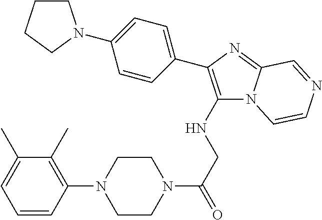 [SMILES: Cc1cccc(N2CCN(C(=O)CNc3c(-c4ccc(N5CCCC5)cc4)nc4cnccn34)CC2)c1C] | 510.3 |
| 257 | 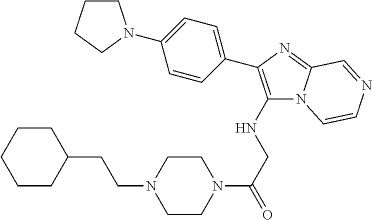 [SMILES: O=C(CNc1c(-c2ccc(N3CCCC3)cc2)nc2cnccn12)N1CCN(CCC2CCCCC2)CC1] | — |
| 258 | 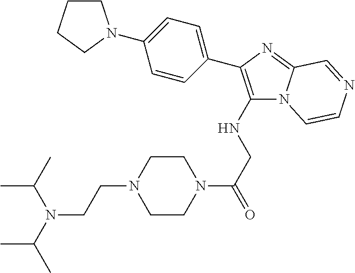 [SMILES: CC(C)N(CCN1CCN(C(=O)CNc2c(-c3ccc(N4CCCC4)cc3)nc3cnccn23)CC1)C(C)C] | — |
| 259 | 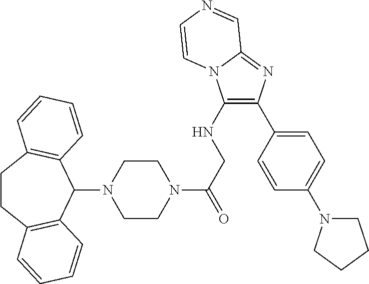 [SMILES: O=C(CNc1c(-c2ccc(N3CCCC3)cc2)nc2cnccn12)N1CCN(C2c3ccccc3CCc3ccccc32)CC1] | — |
| 260 | 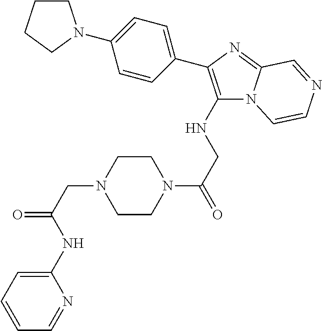 [SMILES: O=C(CN1CCN(C(=O)CNc2c(-c3ccc(N4CCCC4)cc3)nc3cnccn23)CC1)Nc1ccccn1] | — |
| 261 | 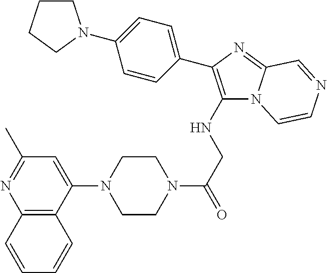 [SMILES: Cc1cc(N2CCN(C(=O)CNc3c(-c4ccc(N5CCCC5)cc4)nc4cnccn34)CC2)c2ccccc2n1] | — |
| 262 | 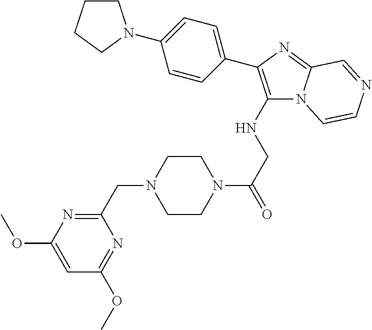 [SMILES: COc1cc(OC)nc(CN2CCN(C(=O)CNc3c(-c4ccc(N5CCCC5)cc4)nc4cnccn34)CC2)n1] | — |
| 263 | 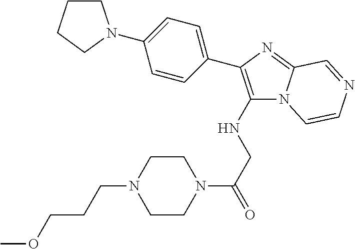 [SMILES: COCCCN1CCN(C(=O)CNc2c(-c3ccc(N4CCCC4)cc3)nc3cnccn23)CC1] | — |
| 264 | 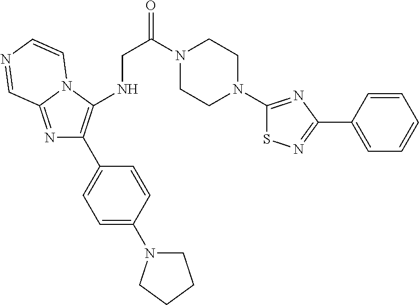 [SMILES: O=C(CNc1c(-c2ccc(N3CCCC3)cc2)nc2cnccn12)N1CCN(c2nc(-c3ccccc3)ns2)CC1] | — |
| 265 | 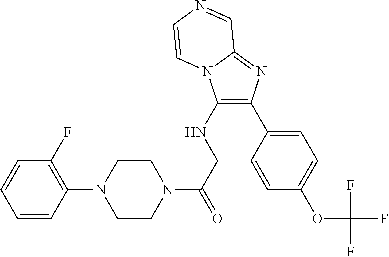 [SMILES: O=C(CNc1c(-c2ccc(OC(F)(F)F)cc2)nc2cnccn12)N1CCN(c2ccccc2F)CC1] | — |
| 266 | 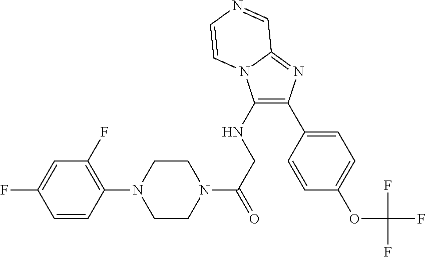 [SMILES: O=C(CNc1c(-c2ccc(OC(F)(F)F)cc2)nc2cnccn12)N1CCN(c2ccc(F)cc2F)CC1] | — |
| 267 | 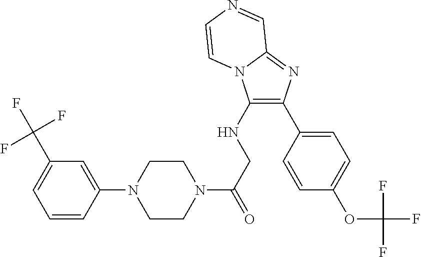 [SMILES: O=C(CNc1c(-c2ccc(OC(F)(F)F)cc2)nc2cnccn12)N1CCN(c2cccc(C(F)(F)F)c2)CC1] | — |
| 268 | 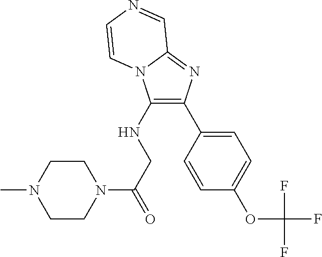 [SMILES: CN1CCN(C(=O)CNc2c(-c3ccc(OC(F)(F)F)cc3)nc3cnccn23)CC1] | — |
| 269 | 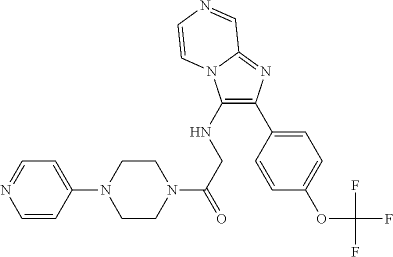 [SMILES: O=C(CNc1c(-c2ccc(OC(F)(F)F)cc2)nc2cnccn12)N1CCN(c2ccncc2)CC1] | — |
| 270 | 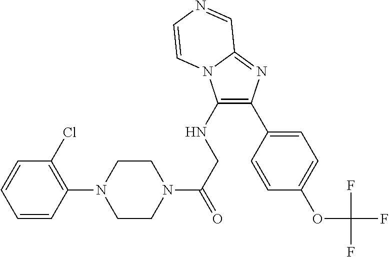 [SMILES: O=C(CNc1c(-c2ccc(OC(F)(F)F)cc2)nc2cnccn12)N1CCN(c2ccccc2Cl)CC1] | — |
| 271 | 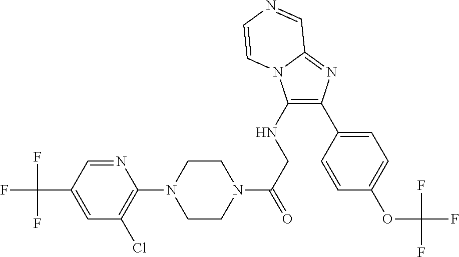 [SMILES: O=C(CNc1c(-c2ccc(OC(F)(F)F)cc2)nc2cnccn12)N1CCN(c2ncc(C(F)(F)F)cc2Cl)CC1] | — |
| 272 | 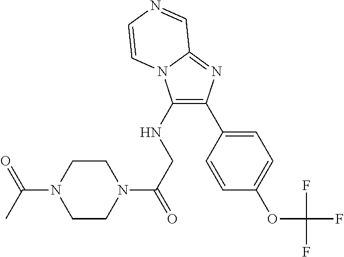 [SMILES: CC(=O)N1CCN(C(=O)CNc2c(-c3ccc(OC(F)(F)F)cc3)nc3cnccn23)CC1] | — |
| 273 | 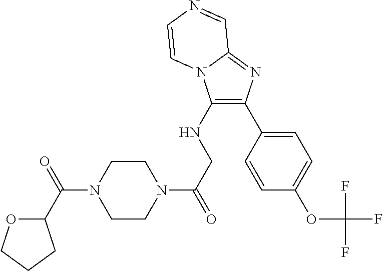 [SMILES: O=C(CNc1c(-c2ccc(OC(F)(F)F)cc2)nc2cnccn12)N1CCN(C(=O)C2CCCO2)CC1] | — |
| 274 | 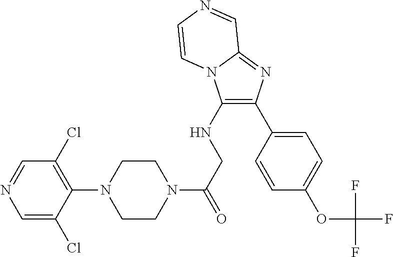 [SMILES: O=C(CNc1c(-c2ccc(OC(F)(F)F)cc2)nc2cnccn12)N1CCN(c2c(Cl)cncc2Cl)CC1] | 566.3 |
| 275 | 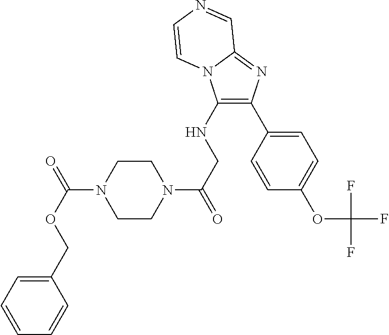 [SMILES: O=C(CNc1c(-c2ccc(OC(F)(F)F)cc2)nc2cnccn12)N1CCN(C(=O)OCc2ccccc2)CC1] | — |
| 276 | 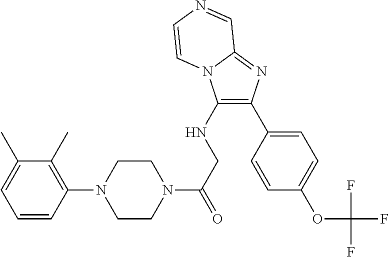 [SMILES: Cc1cccc(N2CCN(C(=O)CNc3c(-c4ccc(OC(F)(F)F)cc4)nc4cnccn34)CC2)c1C] | — |
| 277 | 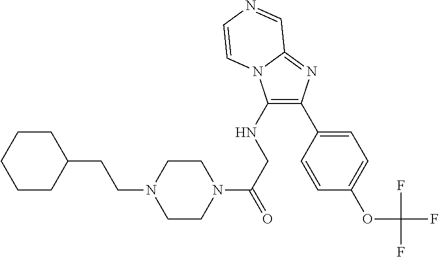 [SMILES: O=C(CNc1c(-c2ccc(OC(F)(F)F)cc2)nc2cnccn12)N1CCN(CCC2CCCCC2)CC1] | |
| 278 | 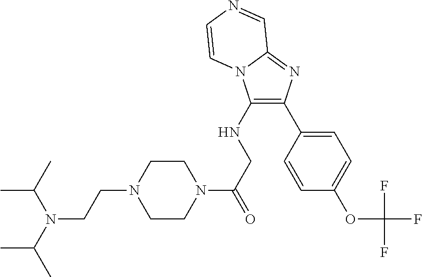 [SMILES: CC(C)N(CCN1CCN(C(=O)CNc2c(-c3ccc(OC(F)(F)F)cc3)nc3cnccn23)CC1)C(C)C] | — |
| 279 | 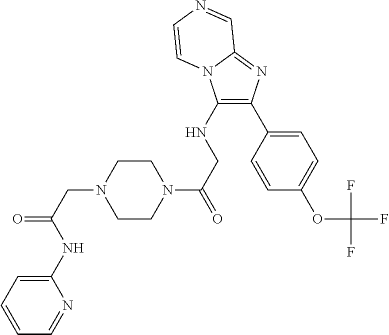 [SMILES: O=C(CN1CCN(C(=O)CNc2c(-c3ccc(OC(F)(F)F)cc3)nc3cnccn23)CC1)Nc1ccccn1] | — |
| 280 | 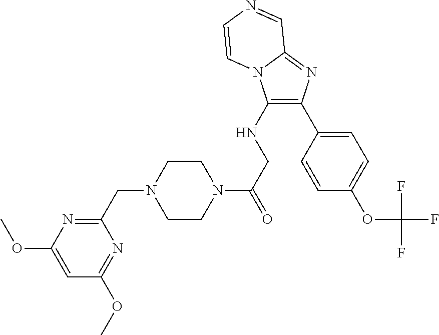 [SMILES: COc1cc(OC)nc(CN2CCN(C(=O)CNc3c(-c4ccc(OC(F)(F)F)cc4)nc4cnccn34)CC2)n1] | — |
| 281 | 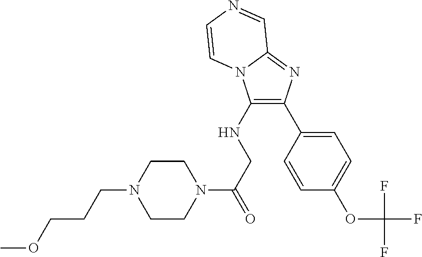 [SMILES: COCCCN1CCN(C(=O)CNc2c(-c3ccc(OC(F)(F)F)cc3)nc3cnccn23)CC1] | — |
| 282 | 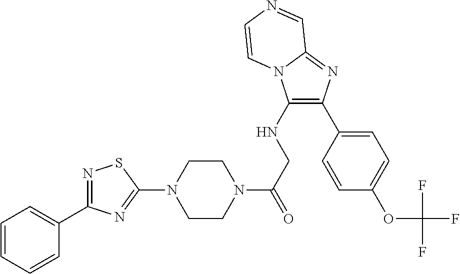 [SMILES: O=C(CNc1c(-c2ccc(OC(F)(F)F)cc2)nc2cnccn12)N1CCN(c2nc(-c3ccccc3)ns2)CC1] | — |
| 283 | 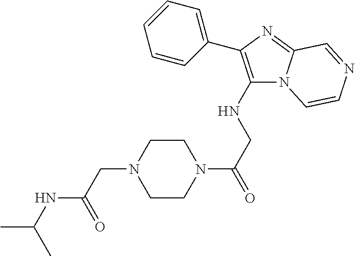 [SMILES: CC(C)NC(=O)CN1CCN(C(=O)CNc2c(-c3ccccc3)nc3cnccn23)CC1] | — |
| 284 | 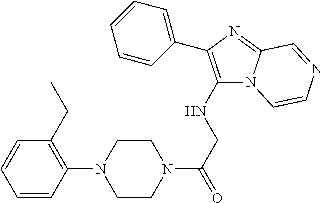 [SMILES: CCc1ccccc1N1CCN(C(=O)CNc2c(-c3ccccc3)nc3cnccn23)CC1] | — |
| 285 | 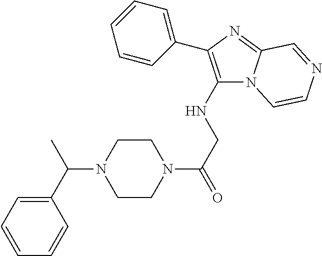 [SMILES: CC(c1ccccc1)N1CCN(C(=O)CNc2c(-c3ccccc3)nc3cnccn23)CC1] | — |
| 286 | 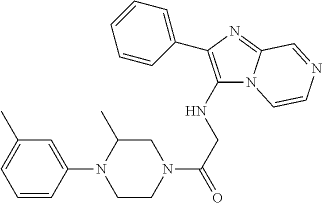 [SMILES: Cc1cccc(N2CCN(C(=O)CNc3c(-c4ccccc4)nc4cnccn34)CC2C)c1] | — |
| 287 | 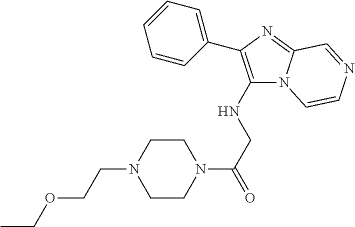 [SMILES: CCOCCN1CCN(C(=O)CNc2c(-c3ccccc3)nc3cnccn23)CC1] | — |
| 288 | 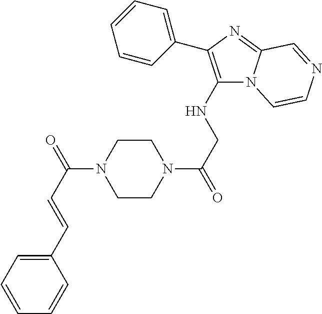 [SMILES: O=C(/C=C/c1ccccc1)N1CCN(C(=O)CNc2c(-c3ccccc3)nc3cnccn23)CC1] | — |
| 289 | 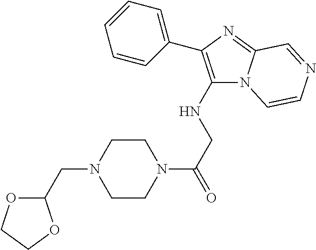 [SMILES: O=C(CNc1c(-c2ccccc2)nc2cnccn12)N1CCN(CC2OCCO2)CC1] | — |
| 290 | 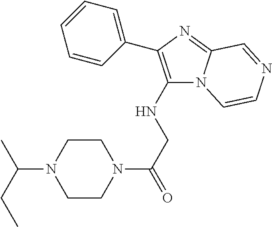 [SMILES: CCC(C)N1CCN(C(=O)CNc2c(-c3ccccc3)nc3cnccn23)CC1] | — |
| 291 | 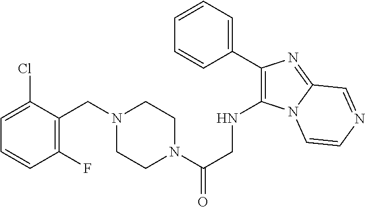 [SMILES: O=C(CNc1c(-c2ccccc2)nc2cnccn12)N1CCN(Cc2c(F)cccc2Cl)CC1] | — |
| 292 | 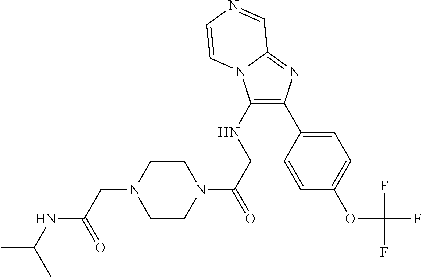 [SMILES: CC(C)NC(=O)CN1CCN(C(=O)CNc2c(-c3ccc(OC(F)(F)F)cc3)nc3cnccn23)CC1] | — |
| 293 | 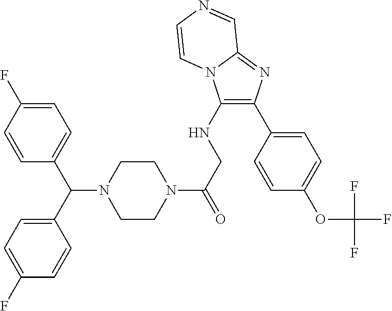 [SMILES: O=C(CNc1c(-c2ccc(OC(F)(F)F)cc2)nc2cnccn12)N1CCN(C(c2ccc(F)cc2)c2ccc(F)cc2)CC1] | — |
| 294 | 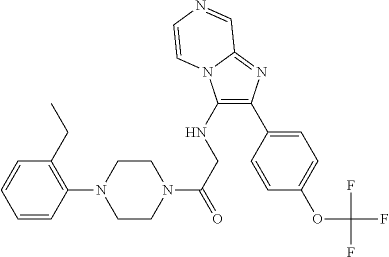 [SMILES: CCc1ccccc1N1CCN(C(=O)CNc2c(-c3ccc(OC(F)(F)F)cc3)nc3cnccn23)CC1] | — |
| 295 | 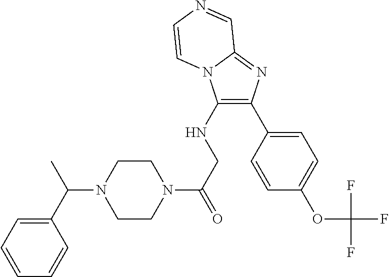 [SMILES: CC(c1ccccc1)N1CCN(C(=O)CNc2c(-c3ccc(OC(F)(F)F)cc3)nc3cnccn23)CC1] | — |
| 296 | 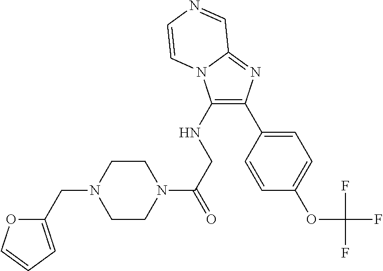 [SMILES: O=C(CNc1c(-c2ccc(OC(F)(F)F)cc2)nc2cnccn12)N1CCN(Cc2ccco2)CC1] | — |
| 297 | 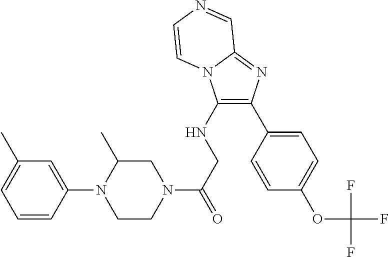 [SMILES: Cc1cccc(N2CCN(C(=O)CNc3c(-c4ccc(OC(F)(F)F)cc4)nc4cnccn34)CC2C)c1] | — |
| 298 | 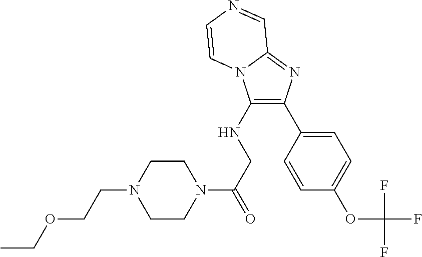 [SMILES: CCOCCN1CCN(C(=O)CNc2c(-c3ccc(OC(F)(F)F)cc3)nc3cnccn23)CC1] | — |
| 299 | 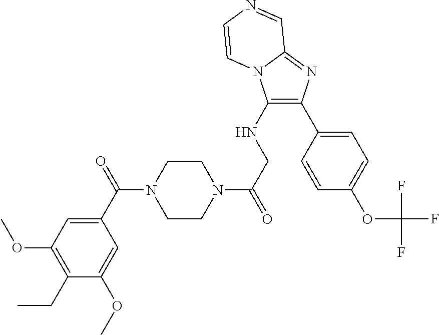 [SMILES: CCc1c(OC)cc(C(=O)N2CCN(C(=O)CNc3c(-c4ccc(OC(F)(F)F)cc4)nc4cnccn34)CC2)cc1OC] | — |
| 300 | 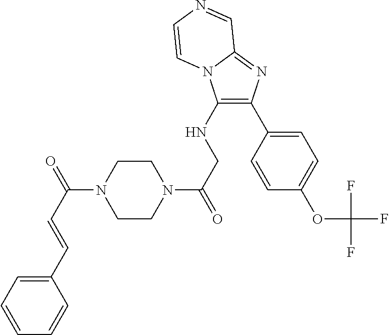 [SMILES: O=C(/C=C/c1ccccc1)N1CCN(C(=O)CNc2c(-c3ccc(OC(F)(F)F)cc3)nc3cnccn23)CC1] | — |
| 301 | 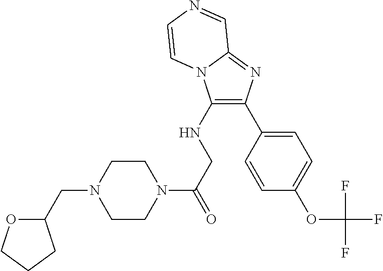 [SMILES: O=C(CNc1c(-c2ccc(OC(F)(F)F)cc2)nc2cnccn12)N1CCN(CC2CCCO2)CC1] | — |
| 302 | 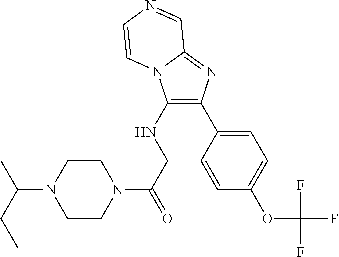 [SMILES: CCC(C)N1CCN(C(=O)CNc2c(-c3ccc(OC(F)(F)F)cc3)nc3cnccn23)CC1] | — |
| 303 | 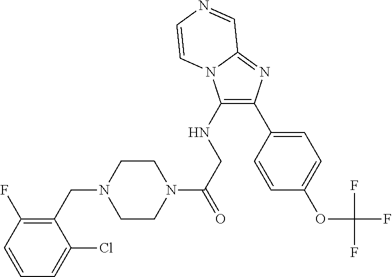 [SMILES: O=C(CNc1c(-c2ccc(OC(F)(F)F)cc2)nc2cnccn12)N1CCN(Cc2c(F)cccc2Cl)CC1] | — |
| 304 | 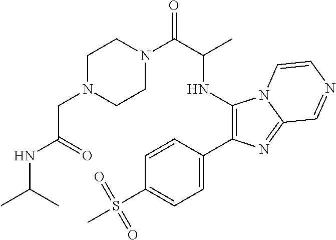 [SMILES: CC(C)NC(=O)CN1CCN(C(=O)C(C)Nc2c(-c3ccc(S(C)(=O)=O)cc3)nc3cnccn23)CC1] | — |
| 305 | 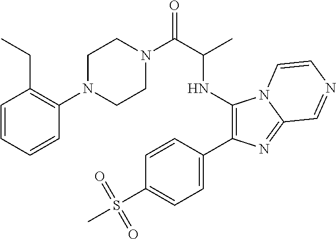 [SMILES: CCc1ccccc1N1CCN(C(=O)C(C)Nc2c(-c3ccc(S(C)(=O)=O)cc3)nc3cnccn23)CC1] | — |
| 306 | 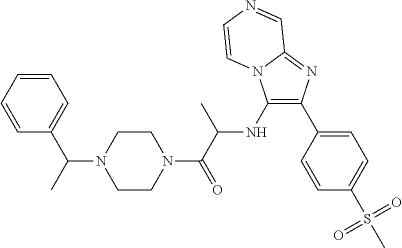 [SMILES: CC(Nc1c(-c2ccc(S(C)(=O)=O)cc2)nc2cnccn12)C(=O)N1CCN(C(C)c2ccccc2)CC1] | — |
| 307 | 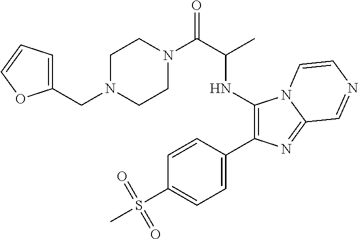 [SMILES: CC(Nc1c(-c2ccc(S(C)(=O)=O)cc2)nc2cnccn12)C(=O)N1CCN(Cc2ccco2)CC1] | — |
| 308 | 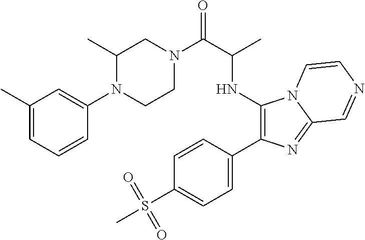 [SMILES: Cc1cccc(N2CCN(C(=O)C(C)Nc3c(-c4ccc(S(C)(=O)=O)cc4)nc4cnccn34)CC2C)c1] | — |
| 309 | 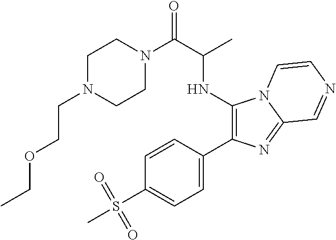 [SMILES: CCOCCN1CCN(C(=O)C(C)Nc2c(-c3ccc(S(C)(=O)=O)cc3)nc3cnccn23)CC1] | — |
| 310 | 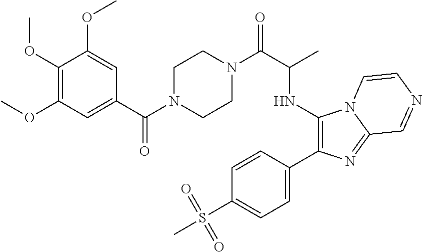 [SMILES: COc1cc(C(=O)N2CCN(C(=O)C(C)Nc3c(-c4ccc(S(C)(=O)=O)cc4)nc4cnccn34)CC2)cc(OC)c1OC] | — |
| 311 | 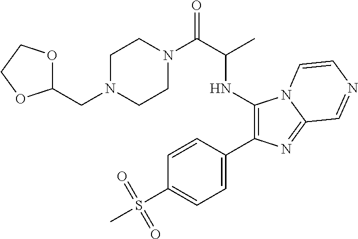 [SMILES: CC(Nc1c(-c2ccc(S(C)(=O)=O)cc2)nc2cnccn12)C(=O)N1CCN(CC2OCCO2)CC1] | — |
| 312 | 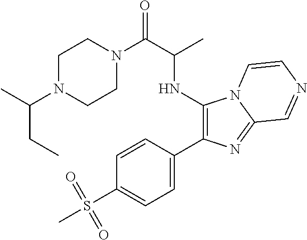 [SMILES: CCC(C)N1CCN(C(=O)C(C)Nc2c(-c3ccc(S(C)(=O)=O)cc3)nc3cnccn23)CC1] | — |
| 313 | 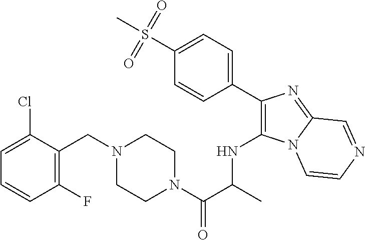 [SMILES: CC(Nc1c(-c2ccc(S(C)(=O)=O)cc2)nc2cnccn12)C(=O)N1CCN(Cc2c(F)cccc2Cl)CC1] | — |
| 314 | 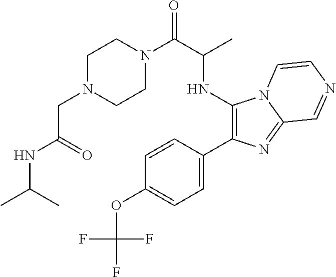 [SMILES: CC(C)NC(=O)CN1CCN(C(=O)C(C)Nc2c(-c3ccc(OC(F)(F)F)cc3)nc3cnccn23)CC1] | — |
| 315 | 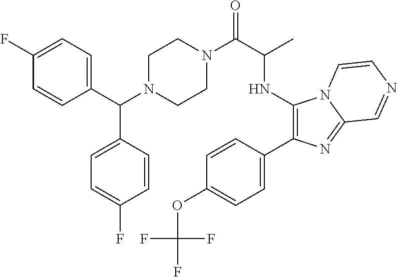 [SMILES: CC(Nc1c(-c2ccc(OC(F)(F)F)cc2)nc2cnccn12)C(=O)N1CCN(C(c2ccc(F)cc2)c2ccc(F)cc2)CC1] | — |
| 316 | 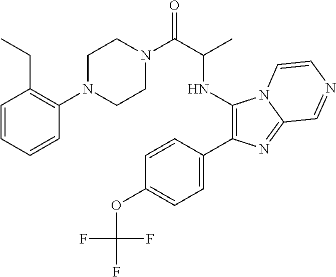 [SMILES: CCc1ccccc1N1CCN(C(=O)C(C)Nc2c(-c3ccc(OC(F)(F)F)cc3)nc3cnccn23)CC1] | — |
| 317 | 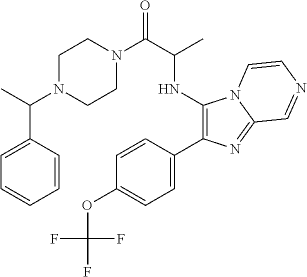 [SMILES: CC(Nc1c(-c2ccc(OC(F)(F)F)cc2)nc2cnccn12)C(=O)N1CCN(C(C)c2ccccc2)CC1] | — |
| 318 | 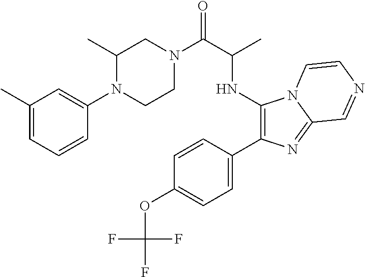 [SMILES: Cc1cccc(N2CCN(C(=O)C(C)Nc3c(-c4ccc(OC(F)(F)F)cc4)nc4cnccn34)CC2C)c1] | — |
| 319 | 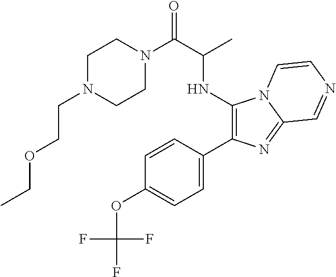 [SMILES: CCOCCN1CCN(C(=O)C(C)Nc2c(-c3ccc(OC(F)(F)F)cc3)nc3cnccn23)CC1] | — |
| 320 | 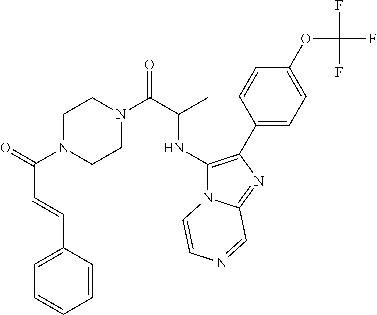 [SMILES: CC(Nc1c(-c2ccc(OC(F)(F)F)cc2)nc2cnccn12)C(=O)N1CCN(C(=O)/C=C/c2ccccc2)CC1] | — |
| 321 | 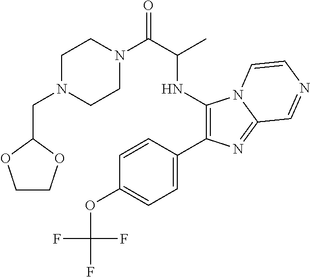 [SMILES: CC(Nc1c(-c2ccc(OC(F)(F)F)cc2)nc2cnccn12)C(=O)N1CCN(CC2OCCO2)CC1] | — |
| 322 | 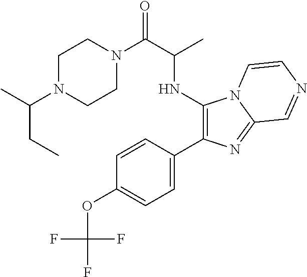 [SMILES: CCC(C)N1CCN(C(=O)C(C)Nc2c(-c3ccc(OC(F)(F)F)cc3)nc3cnccn23)CC1] | — |
| 323 | 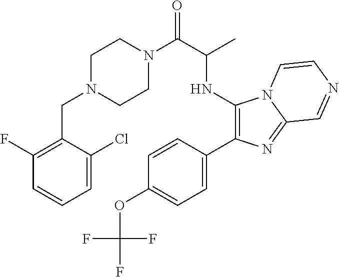 [SMILES: CC(Nc1c(-c2ccc(OC(F)(F)F)cc2)nc2cnccn12)C(=O)N1CCN(Cc2c(F)cccc2Cl)CC1] | — |
| 324 | 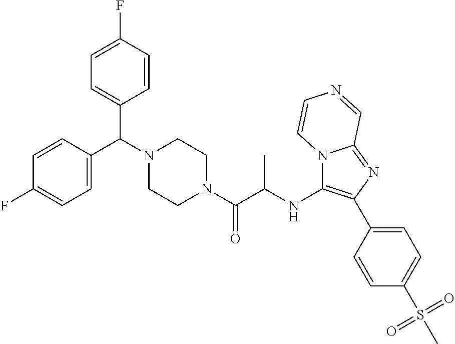 [SMILES: CC(Nc1c(-c2ccc(S(C)(=O)=O)cc2)nc2cnccn12)C(=O)N1CCN(C(c2ccc(F)cc2)c2ccc(F)cc2)CC1] | — |
| 325 | 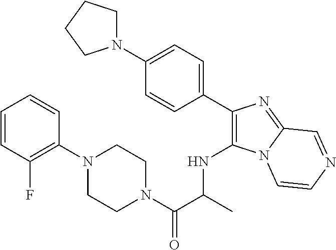 [SMILES: CC(Nc1c(-c2ccc(N3CCCC3)cc2)nc2cnccn12)C(=O)N1CCN(c2ccccc2F)CC1] | — |
| 326 | 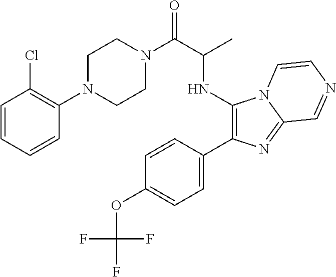 [SMILES: CC(Nc1c(-c2ccc(OC(F)(F)F)cc2)nc2cnccn12)C(=O)N1CCN(c2ccccc2Cl)CC1] | — |
| 327 | 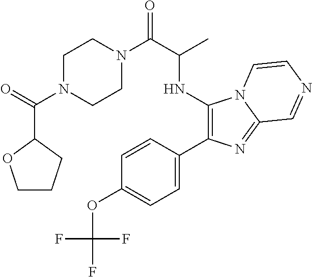 [SMILES: CC(Nc1c(-c2ccc(OC(F)(F)F)cc2)nc2cnccn12)C(=O)N1CCN(C(=O)C2CCCO2)CC1] | — |
| 328 | 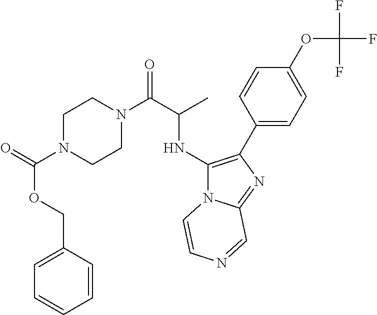 [SMILES: CC(Nc1c(-c2ccc(OC(F)(F)F)cc2)nc2cnccn12)C(=O)N1CCN(C(=O)OCc2ccccc2)CC1] | — |
| 329 | 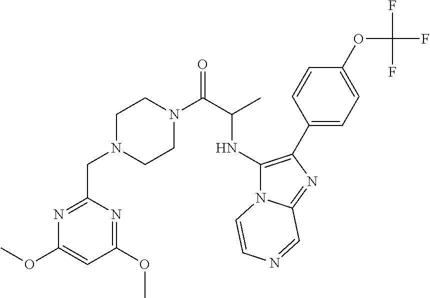 [SMILES: COc1cc(OC)nc(CN2CCN(C(=O)C(C)Nc3c(-c4ccc(OC(F)(F)F)cc4)nc4cnccn34)CC2)n1] | — |
| 330 | 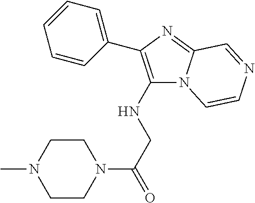 [SMILES: CN1CCN(C(=O)CNc2c(-c3ccccc3)nc3cnccn23)CC1] | — |
| 331 | 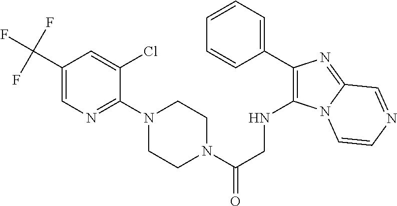 [SMILES: O=C(CNc1c(-c2ccccc2)nc2cnccn12)N1CCN(c2ncc(C(F)(F)F)cc2Cl)CC1] | — |
| 332 | 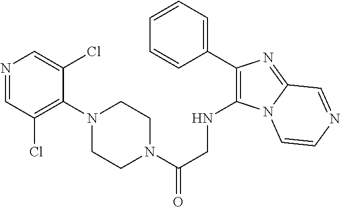 [SMILES: O=C(CNc1c(-c2ccccc2)nc2cnccn12)N1CCN(c2c(Cl)cncc2Cl)CC1] | — |
| 333 | 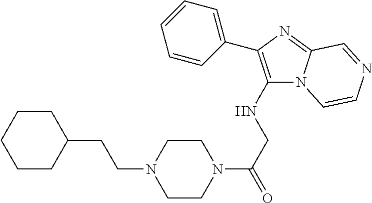 [SMILES: O=C(CNc1c(-c2ccccc2)nc2cnccn12)N1CCN(CCC2CCCCC2)CC1] | — |
| 334 | 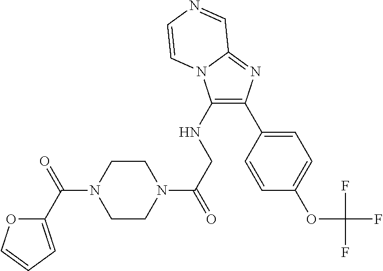 [SMILES: O=C(CNc1c(-c2ccc(OC(F)(F)F)cc2)nc2cnccn12)N1CCN(C(=O)c2ccco2)CC1] | — |
| 335 | 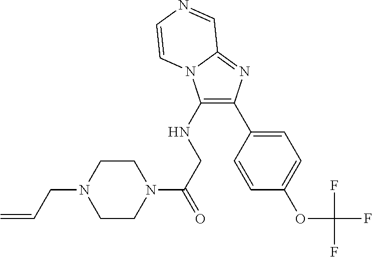 [SMILES: C=CCN1CCN(C(=O)CNc2c(-c3ccc(OC(F)(F)F)cc3)nc3cnccn23)CC1] | — |
| 336 | 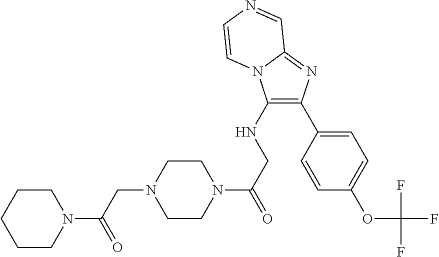 [SMILES: O=C(CNc1c(-c2ccc(OC(F)(F)F)cc2)nc2cnccn12)N1CCN(CC(=O)N2CCCCC2)CC1] | — |
| 337 | 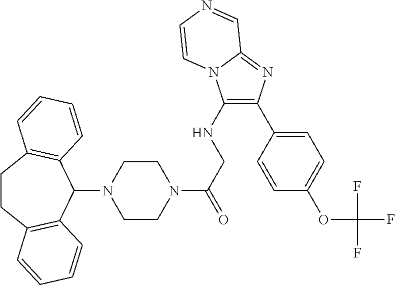 [SMILES: O=C(CNc1c(-c2ccc(OC(F)(F)F)cc2)nc2cnccn12)N1CCN(C2c3ccccc3CCc3ccccc32)CC1] | — |
| 338 | 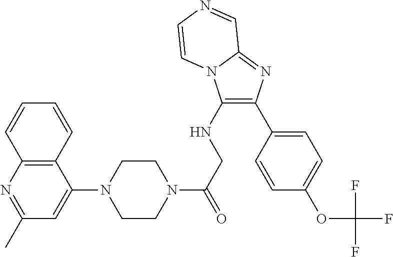 [SMILES: Cc1cc(N2CCN(C(=O)CNc3c(-c4ccc(OC(F)(F)F)cc4)nc4cnccn34)CC2)c2ccccc2n1] | — |
| 339 | 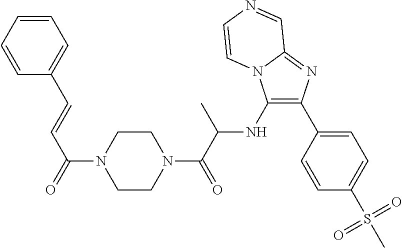 [SMILES: CC(Nc1c(-c2ccc(S(C)(=O)=O)cc2)nc2cnccn12)C(=O)N1CCN(C(=O)/C=C/c2ccccc2)CC1] | — |

### ASSAYS

Compounds useful in the method of the invention were investigated for their utility as antimicrobial agents in the following assay.

Susceptibility testing (MIC determinations) was performed using the standard broth microdilution methodology precisely as described in the Clinical Laboratory Standards Institute Document M7-A6; Methods for Dilution Antimicrobial Susceptibility Tests for Bacteria That Grow Aerobically; Approved Standard- Sixth Edition (ISBN 1-56238-486-4), CLSI, 940 West Valley Road, Suite 1400, Wayne, Pa. 19087-1898 USA. To perform the test, a series of 0.1 ml media in a 96-well plate is prepared with Mueller

Hinton broth to which various concentrations of the test compound are added. The media are then inoculated with a standardized suspension of the test organism, Staphylococcus aureus ATCC29213. After overnight incubation at 35° C., the tests are examined and the minimal inhibitory concentration (MIC) is determined. MIC is defined as the lowest concentration of an antimicrobial agent that prevents visible growth of a microorganism in the broth microdilution susceptibility test

Table 2 contains a list of exemplary compounds which were tested in the above assay. They exhibited MIC values of less than or equal to 8 μg/ml to as low as 0.06 μg/ml.

TABLE 2

| | MIC | | MIC |
|---|---|---|---|
| | Staph. aureus | | Staph. aureus |
| | ATCC29213 | | ATCC29213 |
| ID | 3 μg/ml | ID | 3 μg/ml |
| 26 | 2 | 31 | 0.08 |
| 102 | 1.25 | 184 | 5 |
| 189 | 5 | 268 | 10 |
| 269 | 1.25 | 270 | 10 |
| 274 | 0.08 | 275 | 10 |
| 276 | 10 | 277 | 2.5 |
| 280 | 10 | 281 | 5 |
| 282 | 5 | 295 | 10 |
| 318 | 10 | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each and every document referred to in this patent application is incorporated herein by reference in its entirety for all purposes.

1. A method of a treating a microbial infection, comprising administering to a patient in need of such treatment a compound of Formula I: 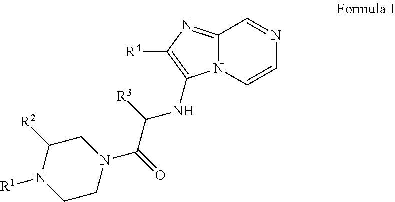
or a pharmaceutically acceptable salt thereof, wherein:
R¹ is methyl, 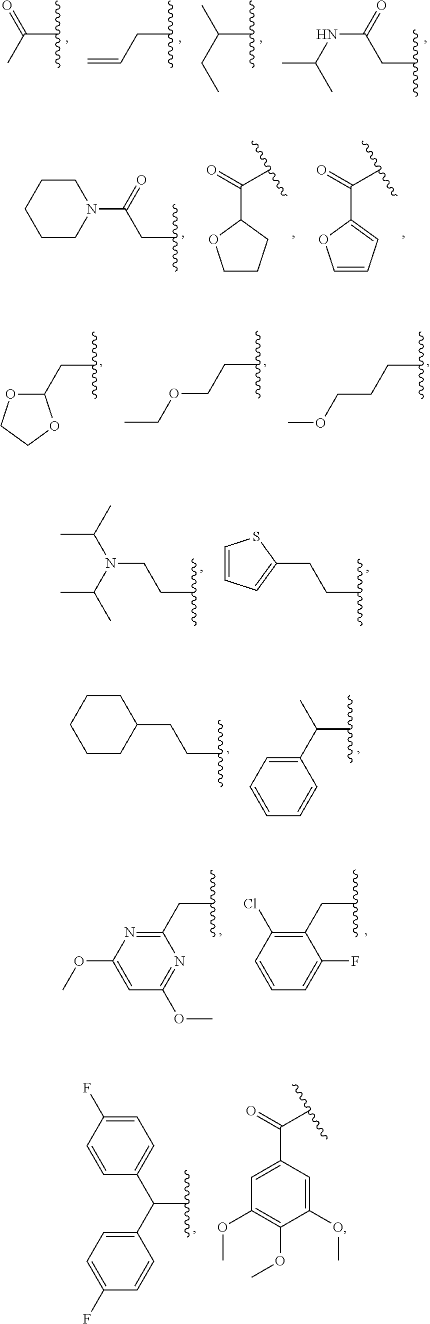 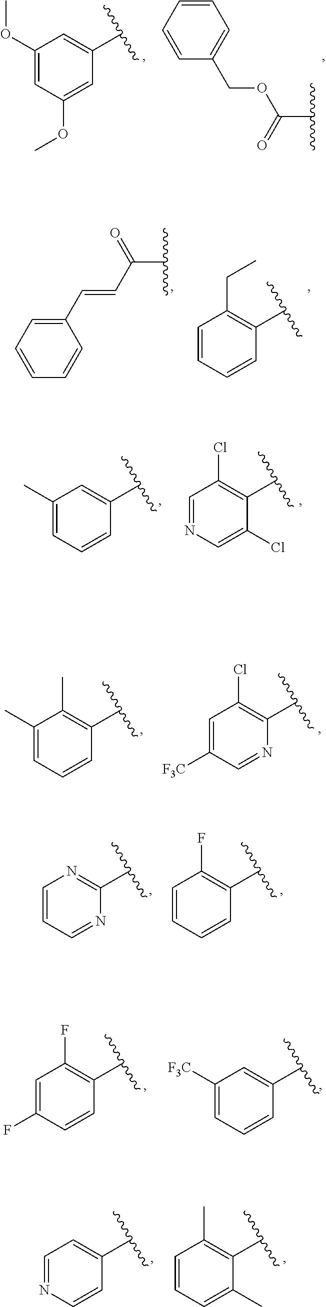 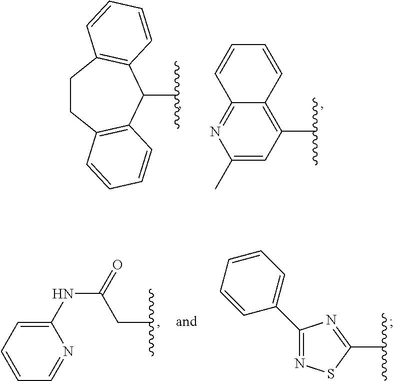
R² is hydrogen or methyl;
R³ is hydrogen or methyl; and
R⁴ is

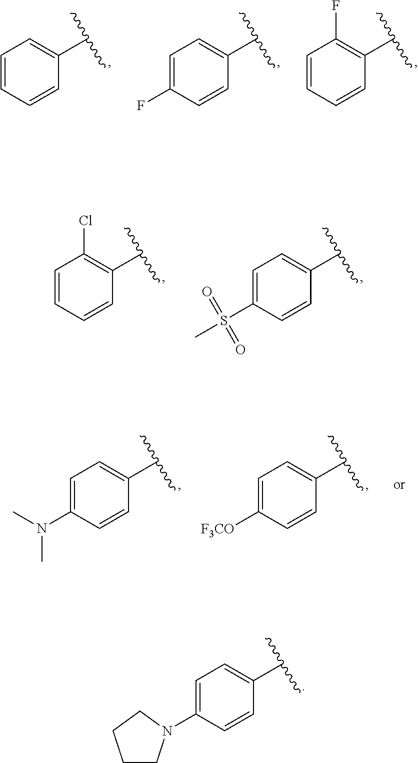

2. The method of claim 1, wherein said microbial infection is a bacterial or fungal infection.

3. The method of claim 2, wherein the bacterial infection is caused by a drug-resistant bacterium.

4. The method of claim 2, wherein the bacterial infection is caused by a gram-negative bacterium.

5. The method of claim 3, wherein the bacterial infection is caused by a gram-positive bacterium.

6. The method of claim 4, wherein said bacterial infection is caused by at least one gram-negative bacterium selected from the group consisting of Acinetobacter spp., Actinobacillus spp., Aeromonas spp., Alcaligenes spp., Bacteroides spp., Bartonella spp., Bordetella spp., Branhamella spp., Brucella spp., Burkholderia spp., Campylobacter spp., Citrobacter spp., Coxiella spp., Edwarsiella spp., Ehrlichia spp., Eikenella spp., Enterobacter spp., Escherichia spp., Flavobacterium spp., Francisella spp., Fusobacterium spp., Haemophilus spp., Haemophilus spp., Helicobacter spp., Kingella spp., Klebsiella spp., Legionella spp., Moraxella spp., Morganella spp., Neisseria spp., Pasteurella spp., Plesiomonas spp., Porphyromonas spp., Prevotella spp., Prevotella spp., Prevotella spp., Proteus spp., Providencia spp., Pseudomonas spp., Ricketsia spp., Salmonella spp., Serratia spp., Shigella spp., Stenotrophomonas spp., Streptobacillus spp., Vibrio spp. and Yersinia spp..

7. The method of claim 6, wherein said bacterial infection is caused by at least one gram-negative organism selected from the group consisting of Acinetobacter baumannii, Pseudomonas aeruginosa, Escherichia coli, Neisseria gonorrhoeae and Chlamydia trachomatis.

8. The method of claim 5, wherein said bacterial infection is caused by at least one gram-positive organism selected from the group consisting of Bacillus spp., Listeria spp., Staphylococcus spp., Enterococcus spp., Clostridium spp., Streptococcus spp., Actinomyces spp. and Mycobacterium spp..

9. The method of claim 8, wherein said bacterial infection is caused by at least one gram-positive organism selected from the group consisting of Staphylococcus aureus, Streptococcus pyogenes, Streptococcus pneumoniae, Clostridium difficile, Enterococcus Faecalis, and Enterococcus faecium.

10. The method of claim 9, wherein said bacterial infection is caused by Staphylococcus aureus.

11. The method of claim 2, wherein the bacterial infection is selected from one or more of the following: urinary tract infection, a respiratory infection, a surgical wound infection, a central line infection, bacteremia, bronchitis, sinusitis, pneumonia, prostatitis, a skin or soft tissue infection, an intra-abdominal infection, or a bacterial infection of febrile neutropenic patients.

12. The method of claim 10, wherein the compound administered is selected from the group consisting of: 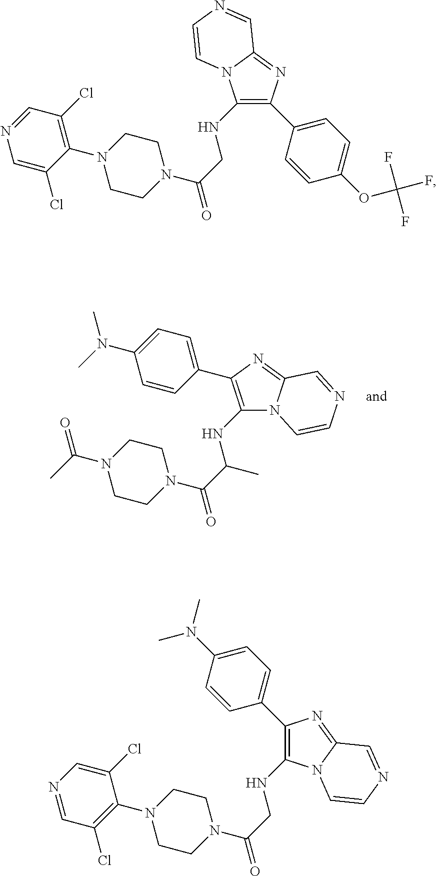 [SMILES: O=C(CNc1c(-c2ccc(OC(F)(F)F)cc2)nc2cnccn12)N1CCN(c2c(Cl)cncc2Cl)CC1 ; CC(=O)N1CCN(C(=O)C(C)Nc2c(-c3ccc(N(C)C)cc3)nc3cnccn23)CC1 ; CN(C)c1ccc(-c2nc3cnccn3c2NCC(=O)N2CCN(c3c(Cl)cncc3Cl)CC2)cc1]
or a pharmaceutically acceptable salt thereof.
